SEQUENCE LISTING (4 sequences)

SEQ ID NO: 1 - protein, 16 aa, Artificial Sequence
  Hypersialylated glycopeptide
  hypersialylated (4)..(4)
YLGNATAIFFLPDEGK

SEQ ID NO: 2 - protein, 30 aa, Artificial Sequence
  Hypersialylated glycopeptide
  hypersialylated (7)..(7)
QLAHQSNSTNIFFSPVSIATAFAMLSLGTK

SEQ ID NO: 3 - protein, 32 aa, Artificial Sequence
  Hypersialylated glycopeptide
  hypersialylated (14)..(14)
ADTHDEILEGLNFNLTEIPEAQIHEGFQELLR

SEQ ID NO: 4 - protein, 394 aa, Artificial Sequence
  human AAT
EDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

(19) United States
(12) Patent Application Publication
(10) Pub. No.: US 2022/0251619 A1
(43) Pub. Date: Aug. 11, 2022

(54) Method of Sialylating a Protein

(71) Applicants: Agency For Science, Technology And Research, Sigapore (SG); The University of Manchester, Manchester (GB)

(72) Inventors: Sern Farh Matthew Choo, Singapore (SG); Terry Nguyen-Khuong, Singapore (SG); Edward George Pallister, Singapore (SG); Say Kong Ng, Singapore (SG); Sabine Lahja Flitsch, Manchester (GB)

(21) Appl. No.: 17/598,965

(22) PCT Filed: Mar. 23, 2020
(86) PCT No.: PCT/SG2020/050154

§ 371 Date: Sep. 28, 2021

(30) Foreign Application Priority Data
Mar. 29, 2019 (SG) 10201902822V

(51) Int. Cl. C12P 21/00; C07K 1/107

(52) U.S. Cl. CPC C12P 21/005; C07K 1/1077

(57) ABSTRACT

The invention relates to a method of increasing the number of α2,3,-α2,6-disialylgalactose N-glycans on a glycoprotein by incubating an α2,3-sialylated glycoprotein with an α2,6-sialyltransferase and a sialic acid source. Also provided is a recombinant glycoprotein comprising at least one α2,3,-α2,6-disialylgalactose N-glycan. In a particular embodiment, the recombinant glycoprotein is alpha-1 antitrypsin (AAT).

### FIELD

The present disclosure relates generally to the field of glycobiology. In particular, the disclosure teaches a method of increasing the number of α2,3-α2,6-disialylgalactose N-glycans on a glycoprotein.

### BACKGROUND

The half-life of a therapeutic glycoprotein can be markedly increased in vivo by increasing the level of sialylation on the glycoprotein. This is believed to be due to negatively charged sialic acid residues that impair interactions between the glycoprotein and hepatic asialoglycoprotein receptors that are present in vivo, which are responsible for endocytic clearance of non-indigenous proteins. The degree of sialylation on a glycoprotein can therefore affect the clearance rate, and serum half-life in the body and is of high clinical relevance. However, methodologies that generate highly sialylated therapeutic glycoproteins are still limited.

An example of a therapeutic glycoprotein is Alpha-1-antitrypisn (AAT). AAT is a protease inhibitor that has a number of roles in the body most notably the inhibition of neutrophil elastase in the lungs. A lack of AAT in the body as a result of the genetic disease AAT deficiency (AATD), causes complications ranging from chronic obstructive pulmonary disease to liver cirrhosis. Augmentation therapy of severe AATD sufferers involves human serum plasma AAT. However, the augmentation treatment is expensive due to AATD patients requiring weekly intravenous treatments, low drug efficiency and limited drug availability. There is therefore a need to develop better AAT therapies with improved efficacies and drug availabilities.

Accordingly, there is a need to overcome, or at least to alleviate, one or more of the above-mentioned problems.

### SUMMARY OF THE INVENTION

Provided herein is an in vitro method, the method comprising a step of incubating an alpha 2,3 sialylated glycoprotein with an alpha 2,6 sialyltransferase and a sialic acid source for a sufficient time and under conditions to increase the number of α2,3,-α2,6-disialylgalactose N-glycans on the glycoprotein as compared to a glycoprotein that has not been incubated with the alpha 2,6 sialyltransferase and sialic acid source.

In one aspect, there is provided a method of increasing sialylation of a glycoprotein, the method comprising a step of incubating an alpha 2,3 sialylated glycoprotein with an alpha 2,6 sialyltransferase and a sialic acid source for a sufficient time and under conditions to increase the number of α2,3,-α2,6-disialylgalactose N-glycans on the glycoprotein as compared to a glycoprotein that has not been incubated with the alpha 2,6 sialyltransferase and sialic acid source.

In one aspect, there is provided a glycoprotein obtained according to a method as defined herein.

In one aspect, there is provided a recombinant glycoprotein comprising at least one α2,3,-α2,6-disialylgalactose N-glycan.

In one aspect, there is provided a composition comprising an alpha 2,6 sialyltransferase and an alpha 2,3 sialyltransferase for increasing the number of α2,3,-α2,6-disialylgalactose N-glycans on a glycoprotein.

In one aspect, there is provided a pharmaceutical composition comprising a glycoprotein as defined herein.

In one aspect, there is provided a glycoprotein as defined herein or a pharmaceutical composition as defined herein for use in therapy.

In one aspect, there is provided a kit for increasing sialylation of a glycoprotein, the kit comprising a column comprising an immobilized α2,6 sialyltransferase.

### BRIEF DESCRIPTION OF THE DRAWINGS

Some embodiments of the present invention will now be described by way of non-limiting example only, with reference to the accompanying drawings in which:

FIG. 1. a) Percent of the total glycan intensity of rAAT that is sialylated after each incubation b) Percent of total glycan intensity of rAAT that contain glycans with 1-5 sialic acids after each incubation. (N.B. Relative Glycan Abundance (±SEM) is calculated as the % contribution to the total glycan intensity of the sample seen in the LC-MS/MS glycopeptide analysis, where the sum of all the glycan intensities=100%)

FIG. 2. Extracted ion chromatogram in the LC-MS/MS at m/z 948 for; untreated (red) and α2,6PTB treated (blue) rAAT during glycopeptide elution (50-100 mins).

FIG. 3. Proposed hypersialylation structures and potential diagnostic fragments of N-Glycans (a) Bisecting NeuSAc (a2-6) sialylation of GlcNAc residue (b) Neu5Ac-a2,8-Neu5Aca2,3-Gal polysialylation (c) Neu5Ac-a2,3-(Neu5Ac-a2,6)-Gal double sialylation of a terminal galactose.

FIG. 4. MALDI-TOF-MS/MS of sialylated N-Glycan species from α2,6PTB treated rAAT (m/z 500-1400) a) HexNAc₄Hexose₅Neu5 Ac₃ b) HexNAc₄Hexose₅Neu5Ac₂ c) HexNAc₄Hexose₅Neu5Ac₁. Green=unique DSG product ion fragments, Orange=Increases in product ion intensities due to presence of DSG product ion fragments.

FIG. 5. LC-MS/MS of hypersialylated glycopeptide (site=YLGNATAIFFLPDEGK (SEQ ID NO: 1)) from α2,6PTB treated rAAT ([M+3H]³⁺=m/z 1466.6329) showing the glycan related fragments.

FIG. 6. Example spectrum of bisecting hypersialylated glycopeptide [(M+4H]⁴⁺=1223.9913) from Fetuin.

FIG. 7. MALDI-TOF-MS of PSLac after ethylation reaction.

FIG. 8. MALDI-TOF-MS/MS of DSLac after ethylation reaction.

FIG. 9. MALDI-TOF-MS/MS of DSlac after ethylation reaction (precursor mass [M+Na]⁺=m/z 957).

FIG. 10. MALDI-TOF-MS/MS of PSlac after ethylation reaction (precursor mass [M+Na]⁺=m/z 911).

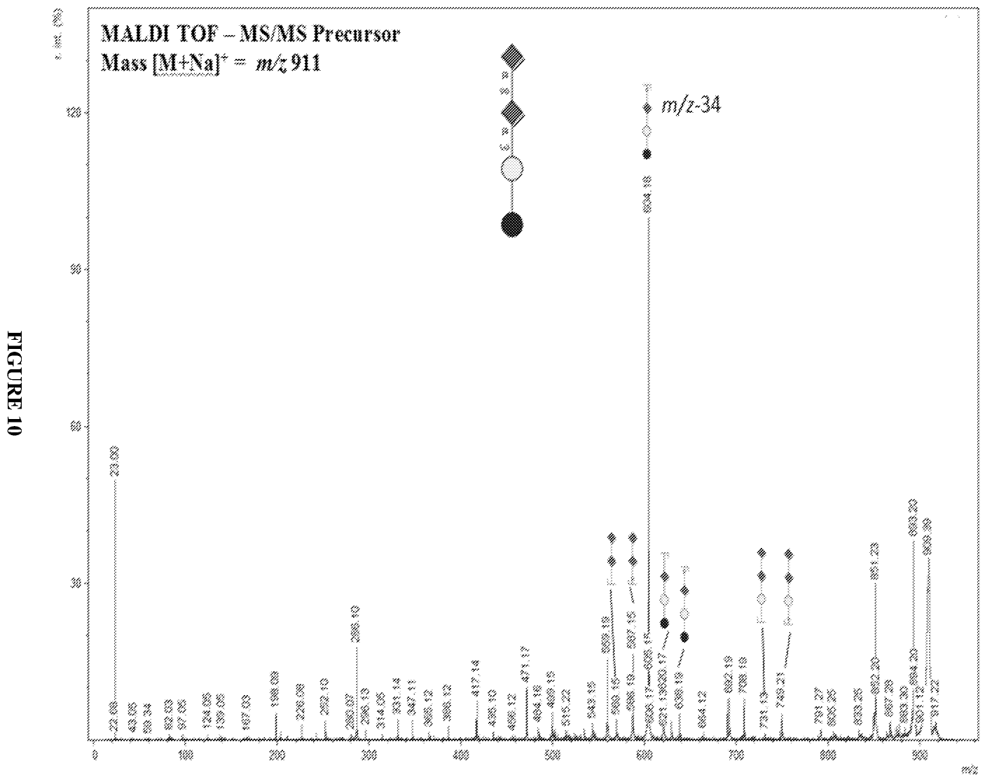

FIG. 11. LC-MS/MS of hypersialylated esterified glycopeptide ([M+3H]=1491.98) showing the glycan related fragments LC-MS/MS of hypersialylated glycopeptide ([M+3H]=1491.98) showing the glycan related fragments.

FIG. 12. 1000-3000 m/z MALDI-TOF-MS of permethylated N-Glycans released from rAAT following α2,6 sialyltransferase Photobacterium damselae treatment.

FIG. 13. 3000-5000 m/z MALDI-TOF-MS of permethylated N-Glycans released from rAAT following α2,6 sialyltransferase Photobacterium damselae treatment.

FIG. 14. LC-MS/MS of hypersialylated glycopeptide (site=QLAHQS [symbol] STNIFFSPVSIATAFAMLSLGTK (SEQ ID NO: 2)) from α2,6PTB treated rAAT ([M+5H]⁵⁺=m/z 1165.5192) showing the glycan related fragments.

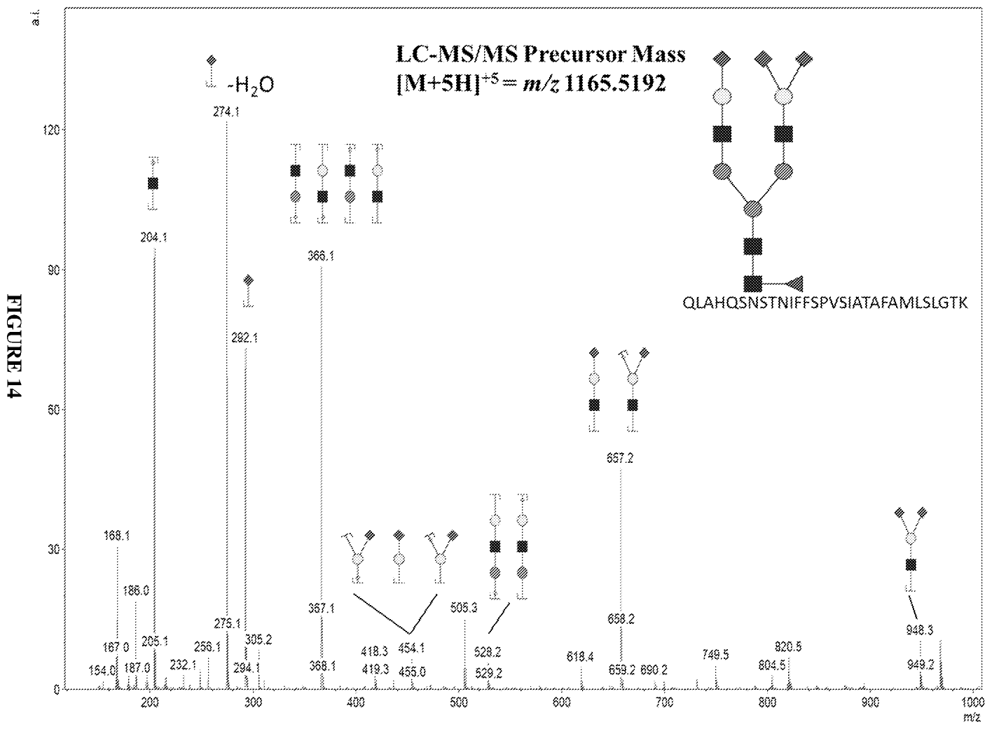

FIG. 15. LC-MS/MS of hypersialylated glycopeptide (site=ADTHDEILEGLNF [symbol] LTEIPEAQIHEGFQELLR (SEQ ID NO: 3)) from α2,6PTB treated rAAT ([M+5H]⁵⁺=m/z 1398.8013) showing the glycan related fragments.

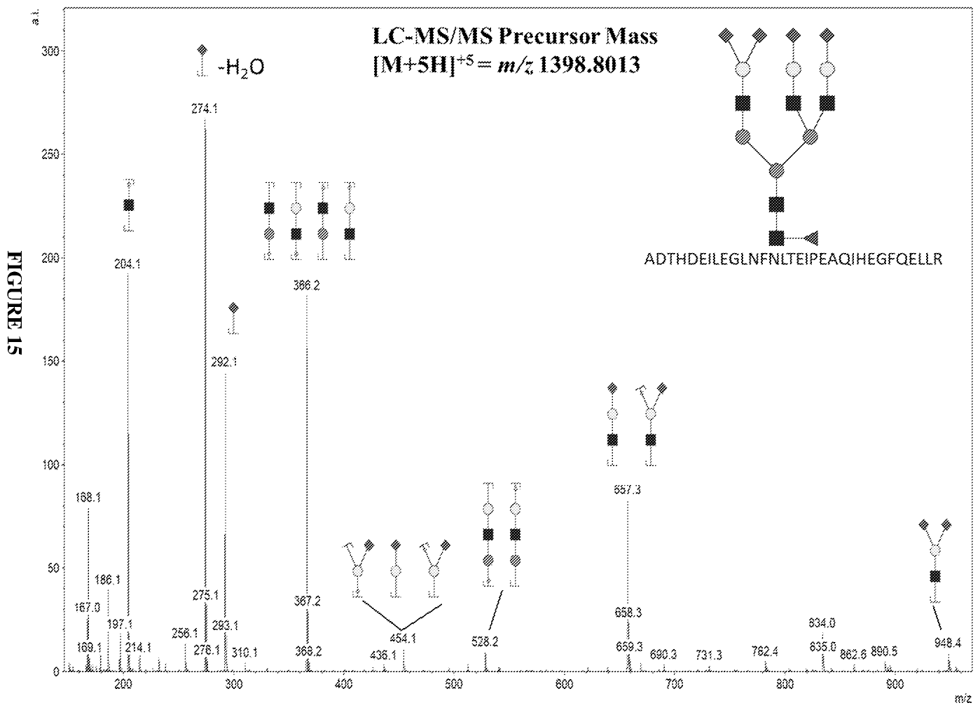

FIG. 16. Chemical structure of the α2,3-α2,6-disialylgalactose ((Neu5Ac-a2,3(Neu5Ac-a2,6)Gal) structure produced on the N-Glycans following the glycan remodeling.

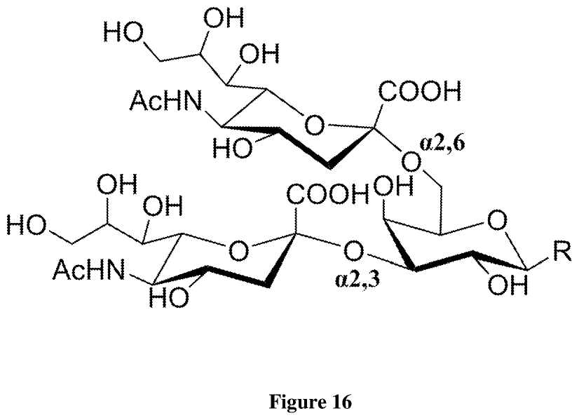

### DETAILED DESCRIPTION

Provided herein is an in vitro method, the method comprising a step of incubating an alpha 2,3 sialylated glycoprotein with an alpha 2,6 sialyltransferase and a sialic acid source.

In one aspect, there is provided an in vitro method, the method comprising a step of incubating an alpha 2,3 sialylated glycoprotein with an alpha 2,6 sialyltransferase and a sialic acid source for a sufficient time and under conditions to increase the number of α2,3,-α2,6-disialylgalactose N-glycans on the glycoprotein as compared to a glycoprotein that has not been incubated with the alpha 2,6 sialyltransferase and sialic acid source.

The method may increase the number of sialic acids upon an N-glycan. For example, the method may increase the number of sialic acids per galactose of the glycans on the glycoprotein.

The term “glycosylation” denotes the chemical reaction of covalently coupling a glycosyl residue to an acceptor group. One specific acceptor group is a hydroxyl group. e.g. a hydroxyl group of another sugar. “Sialylation” is a specific form of glycosylation wherein the acceptor group is reacted with a sialic acid (═N-acetylneuraminic acid) residue. Such a reaction is typically catalyzed by a sialyltransferase enzyme using, for example, cytidine-5′-monophospho-N-acetylneuraminic acid as donor compound or co-substrate.

“Sialylation” is a specific embodiment of a result of glycosyltransferase enzymatic activity (sialyltransferase enzymatic activity in the particular case), under conditions permitting the same. Generally, the skilled person appreciates that the aqueous buffer in which a glycosyltransferase enzymatic reaction can be performed (=“permitting glycosyltransferase enzymatic activity”) needs to be buffered using a buffer salt such as Tris, MES, phosphate, acetate, or another buffer salt specifically capable of buffering in the pH range of pH 6 to pH 8, more specifically in the range of pH 6 to pH 7, even more specifically capable of buffering a solution of about pH 6.5. The buffer may further contain a neutral salt such as but not limited to NaCl. Further, in particular embodiments the skilled person may consider adding to the aqueous buffer a salt comprising a divalent ion such as Mg²⁺ or Mn²⁺, e.g. but not limited to MgCl₂ and MnCl₂. Conditions permitting glycosyltransferase enzymatic activity known to the art include ambient (room) temperature, but more generally temperatures in the range of 0° C. to 40° C. particularly 10° C. to 30° C. particularly 20° C.

The term “glycan” refers to a poly- or oligosaccharide, i.e. to a multimeric compound which upon acid hydrolysis yields a plurality of monosachbarides. A glycoprotein comprises one or more glycan moieties which are covalently coupled to side groups of the polypeptide chain, typically via asparagine or arginine (“N-linked glycosylation”) or via serine or threonine (“O-linked glycosylation”).

As used herein, the term “glycoprotein” refers to a protein that contains a peptide backbone covalently linked to one or more sugar moieties (i.e., glycans). Sugar moiety(ies) may be in the form of disaccharides, oligosaccharides, and/or polysaccharides. Sugar moiety(ies) may comprise a single unbranched chain of sugar residues or may comprise one or more branched chains. Glycoproteins can contain O-linked sugar moieties and/or N-linked sugar moieties.

As used herein, “polypeptide” (or “amino acid sequence” or “protein”) refers to an oligopeptide, peptide, polypeptide, or protein sequence, and fragments or portions thereof, and to naturally occurring or synthetic molecules. “Amino acid sequence” and like terms, such as “polypeptide” or “protein”, are not meant to limit the indicated amino acid sequence to the complete, native amino acid sequence associated with the recited protein molecule.

Any protein as disclosed herein may, in an embodiment, comprise a “protein tag” % which is a peptide sequence genetically grafted onto the recombinant protein. A protein tag may comprise a linker sequence with a specific protease cleavage site to facilitate removal of the tag by proteolysis. As a specific embodiment, an “affinity tag” is appended to a target protein so that the target can be purified from its crude biological source using an affinity technique. For example, the source can be a transformed host organism expressing the target protein or a culture supernatant into which the target protein was secreted by the transformed host organism. Specific embodiments of an affinity tag include chitin binding protein (CBP), maltose binding protein (MBP), and glutathione-S-transferase (GST) The poly(His) tag is a widely-used protein tag which facilitates binding to certain metal chelating matrices.

The term “chimeric protein”, “fusion protein” or “fusion polypeptide” refers to a protein whose amino acid sequence represents a fusion product of subsequences of the amino acid sequences from at least two distinct proteins. A fusion protein typically is not produced by direct manipulation of amino acid sequences, but, rather, is expressed from a “chimeric” gene that encodes the chimeric amino acid sequence.

In one embodiment, the method comprises improving the pharmacokinetics of the therapeutic glycoprotein.

The term “pharmacokinetic property” or “pharmacokinetics” refers to a parameter that describes the disposition of an active agent in an organism or host. Representative pharmacokinetic properties include in vivo (plasma)half-life, clearance, rate of elimination; volume of distribution, degree of tissue targeting, degree of cell type targeting, and the like.

In one embodiment, the pharmacokinetics of the therapeutic glycoprotein is improved as compared to a glycoprotein that has not been incubated with the alpha 2,6 sialyltransferase and sialic acid source.

In one embodiment, the method comprises improving the in vivo half-life of the therapeutic glycoprotein.

The terms “half-life” “in vivo half-life” and “plasma half-life” are used interchangeably, and refers to the time by which half of the administered amount of a molecule, such as a therapeutic glycoprotein, is removed from the blood stream.

In one embodiment, the in vivo half-life of the therapeutic glycoprotein is improved as compared to a glycoprotein that has not been incubated with the alpha 2,6 sialyltransferase and sialic acid source.

In one embodiment, the method comprises altering the sialylation pattern of the therapeutic glycoprotein.

In one embodiment, the method comprises increasing in-vivo sialidase resistance of the glycoprotein.

The term “sialidase-resistant” when used to refer to a glycoprotein, describes the characteristic of being substantially resistant to cleavage by sialidase treatment as defined herein.

In one embodiment, the in vivo sialidase resistance of the therapeutic glycoprotein is improved as compared to a glycoprotein that has not been incubated with the alpha 2,6 sialyltransferase and sialic acid source.

The term “sialic acid” refers to any member of a family of nine-carbon carboxylated sugars. The most common member of the sialic acid family is N-acetyl-neuraminic acid (2-keto-5-acetamido-3,5-dideoxy-D-glycero-D-galactononulopyranos-1-onic acid (often abbreviated as Neu5Ac, Neu5Ac, or NANA). A second member of the family is N-glycolyl-neuraminic acid (Neu5Gc or NeuGc), in which the N-acetyl group of Neu5Ac is hydroxylated. A third sialic acid family member is 2-keto-3-deoxy-nonulosonic acid (KDN). Also included are 9-substituted sialic acids such as a 9-O—C₁-C₆ acyl-Neu5Ac like 9-O-lactyl-Neu5Ac or 9-O-acetyl-Neu5Ac, 9-deoxy-9-fluoro-Neu5Ac and 9-azido-9-deoxy-Neu5Ac.

The sialic acid source may also be referred to as a sialic acid donor. In one embodiment, the sialic acid source is cytidine-monophosphate-N-Acetyl-Neuraminic-Acid. The sialic acid source may also include natural or unnatural sialic acid derivatives such as, but not limited to, cytidine-monophosphate-3-keto-deoxynonic acid, cytidine-monophosphate-N-Glycolylneuraminic acid and cytidine-monophosphate-azido sialic acids

In one embodiment, the α2,6 sialyltransferase is an α2,6 sialyltransferase from a photobacterium.

In one embodiment, the α2,6 sialyltransferase is a purified α2,6 sialyltransferase from photobacterium or is an α2,6 sialyltransferase enzyme extract from photobacterium.

In one embodiment, the photobacterium is Photobacterium damselae.

In one embodiment, the glycoprotein is a recombinant glycoprotein or an isolated naturally-occurring glycoprotein.

The term “isolated” as used herein means altered “by the hand of man” from its natural state; i.e., if it occurs in nature, it has been changed or removed from its original environment, or both. For example, a naturally occurring polypeptide naturally present in a bacterium is not “isolated”, but the same polypeptide separated from the coexisting materials of its natural state is “isolated”, as the term is employed herein.

The term “recombinant” refers to an amino acid sequence or a nucleotide sequence that has been intentionally modified by recombinant methods. By the term “recombinant nucleic acid” herein is meant a nucleic acid, originally formed in vitro, in general, by the manipulation of a nucleic acid by endonucleases, in a form not normally found in nature. A “recombinant protein” or “recombinantly produced protein” is a protein made using recombinant techniques. i.e., through the expression of a recombinant nucleic acid as depicted above.

The terms “nucleic acid” or “polynucleoide” can be used interchangeably and refer to a polymer that can be corresponded to a ribose nucleic acid (RNA) or deoxyribose nucleic acid (DNA) polymer, or an analog thereof. This includes polymers of nucleotides such as RNA and DNA, as well as synthetic forms, modified (e.g., chemically or biochemically modified) forms thereof, and mixed polymers (e.g., including both RNA and DNA subunits).

A nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation.

The term “vector” refers to a piece of DNA, typically double-stranded, which may have inserted into it a piece of foreign DNA. The vector or may be, for example, of plasmid origin. Vectors contain “replicon” polynucleotide sequences that facilitate the autonomous replication of the vector in a host cell. Foreign DNA is defined as heterologous DNA, which is DNA not naturally found in the host cell, which, for example, replicates the vector molecule, encodes a selectable or screenable marker, or encodes a transgene. The vector is used to transport the foreign or heterologous DNA into a suitable host cell. Once in the host cell, the vector can replicate independently of or coincidental with the host chromosomal DNA, and several copies of the vector and its inserted DNA can be generated. In addition, the vector can also contain the necessary elements that permit transcription of the inserted DNA into an mRNA molecule or otherwise cause replication of the inserted DNA into multiple copies of RNA Some expression vectors additionally contain sequence elements adjacent to the inserted DNA that increase the half-life of the expressed mRNA and/or allow translation of the mRNA into a protein molecule. Many molecules of mRNA and polypeptide encoded by the inserted DNA can thus be rapidly synthesized.

The glycoprotein as referred to herein may be expressed in a host cell. The term “host cell” refers to both single-cellular prokaryote and eukaryote organisms (e.g., mammalian cells, insect cells, bacteria, yeast, and actinomycetes) and single cells from higher order plants or animals when being grown in cell culture.

In one embodiment, the glycoprotein is a Chinese Hamster Ovary (CHO) cell expressed glycoprotein.

Proteins that can be modified by the methods of the invention include, for example, hormones such as insulin, growth hormones (including human growth hormone and bovine growth hormone), tissue-type plasminogen activator (t-PA), renin, clotting factors such as factor VIII and factor IX, bombesin, thrombin, hemopoietic growth factor, serum albumin, receptors for hormones or growth factors, interleukins, colony stimulating factors. T-cell receptors, MHC polypeptides, viral antigens, glycosyltransferases, and the like. Polypeptides of interest for recombinant expression and subsequent modification using the methods of the invention also include alpha-1 antitrypsin (AAT), erythropoietin, granulocyte-macrophage colony stimulating factor, anti-thrombin III, interleukin 6, interferon β, protein C, fibrinogen, among many others. This list of polypeptides is exemplary, not exclusive. The methods are also useful for modifying the sialylation patterns of chimeric proteins, including, but not limited to, chimeric proteins that include a moiety derived from an immunoglobulin, such as IgG.

In some embodiments, the disclosure comprises, without limitation, cell surface glycoproteins and glycoproteins present in soluble form in serum (“serum glycoprotein”), the glycoproteins particularly being of mammalian origin. A “cell surface glycoprotein” is understood to be glycoprotein of which a portion is located on and bound to the surface of a membrane, by way of a membrane anchor portion of the surface glycoprotein's polypeptide chain, wherein the membrane is part of a biological cell. The term cell surface glycoprotein also encompasses isolated forms of the cell surface glycoprotein as well as soluble fragments thereof which are separated from the membrane anchor portion, e.g. by proteolytic cleavage or by recombinant production of such soluble fragments. A “serum glycoprotein” is understood as a glycoprotein being present in serum, i.e. a blood protein present in the non-cellular portion of whole blood, e.g. in the supernatant following sedimentation of cellular blood components. Without limitation, a specifically regarded and embodied serum glycoprotein is an immunoglobulin. Particular immunoglobulins mentioned in here belong to the IgG group (characterized by Gamma heavy chains), specifically any of four the IgG subgroups. For the disclosures, aspects and embodiments herein the term “serum glycoprotein also encompasses a monoclonal antibody; monoclonal antibodies artificially are well known to the art and can be produced e.g. by hybridoma cells or recombinantly using transformed host cells. A further serum specific glycoprotein is a carrier protein such as serum albumin, a fetuin, or another glycoprotein member of the superfamily of histidine-rich glycoproteins of which the fetuins are members. Further, without limitation, a specifically regarded and embodied serum glycoprotein regarding all disclosures, aspects and embodiments herein is a glycosylated protein signaling molecule. A particular molecule of this group is erythropoietin (EPO).

In one embodiment, the recombinant glycoprotein is alpha-1 antitrypsin (AAT). In one embodiment, the recombinant glycoprotein is erythropoietin. In another embodiment, the recombinant glycoprotein is an antibody of a fragment thereof. The antibody may, for example, be an antibody-conjugate.

In one embodiment, the recombinant glycoprotein is human AAT having at least 90% identity to the following sequence:

(SEQ ID NO: 4)
EDPQGDAAQKTDTSHHDQDHPTFNKITPNLAEFAFSLYRQL
AHQSNSTNIFFSPVSIATAFAMLSLGTKADTHDEILEGLN
FNLTEIPEAQIHEGFQELLRTLNQPDSQLQLTTGNGLFL
SEGLKLVDKFLEDVKKLYHSEAFTVNFGDTEEAKKQINDY
VEKGTQGKIVDLVKELDRDTVFALVNYIFFKGKWERPFEV
KDTEEEDFHVDQVTTVKVPMMKRLGMFNIQHCKKLSSWVLL
MKYLGNATAIFFLPDEGKLQHLENELTHDIITKFLENEDR
RSASLHLPKLSITGTYDLKSVLGQLGITKVFSNGADLSGVT
EEAPLKLSKAVHKAVLTIDEKGTEAAGAMFLEAIPMSIPPE
VKFNKPFVFLMIEQNTKSPLFMGKVVNPTQK.

The method may comprise a prior or concurrent step of incubating the glycoprotein with an alpha 2,3 sialyltransferase and a sialic acid source for a sufficient time and under conditions to increase alpha 2,3 sialylation of the glycoprotein to a saturation point as compared to a glycoprotein that has not been incubated with an alpha 2,3 sialyltransferase and a sialic acid source.

The method may comprise a prior or concurrent step of incubating the glycoprotein with a β-1,4-galactosyltransferase and a galactose source for a sufficient time and under conditions to increase branching, the elongation and/or galactosylation of the glycoprotein as compared to a glycoprotein that has not been incubated with the β-1,4-galactosyltransferase and a galactose source.

In one aspect, there is provided a method of increasing sialylation of a glycoprotein, the method comprising a step of incubating an alpha 2,3 sialylated glycoprotein with an alpha 2,6 sialyltransferase and a sialic acid source for a sufficient time and under conditions to increase the number of α2,3,-α2,6-disialylgalactose N-glycans on the glycoprotein as compared to a glycoprotein that has not been incubated with the alpha 2,6 sialyltransferase and the sialic acid source.

In one aspect, there is provided a glycoprotein obtained according to a method as defined herein.

In one aspect, there is provided a recombinant glycoprotein comprising at least one α2,3,-α2,6-disialylgalactose N-glycan.

The recombinant glycoprotein may comprises an amino acid sequence of SEQ ID NO: 4, wherein the amino acid sequence of SEQ ID NO:4 comprises an α2,3,-α2,6-disialylgalactose N-glycan at an amino acid position selected from the group consisting of Asn-46, Asn-83 and Asn-247.

The recombinant glycoprotein may have an α2,3,-α2,6-disialylgalactose N-glycan at only one position (e.g. Asn-46, Asn-83 or Asn-247) of SEQ ID NO: 4. The recombinant glycoprotein may also have an α2,3,-α2,6-disialylgalactose N-glycan on two positions (e.g. Asn-46 and Asn-83, Asn-46 and Asn-247 or Asn-83 and Asn-247) of SEQ ID NO: 4. The recombinant glycoprotein may also have an α2,3,-α2,6-disialylgalactose N-glycan on all three positions (Asn-46, Asn-83 and Asn-247) of SEQ ID NO: 4.

In one aspect, there is provided a composition comprising an alpha 2,6 sialyltransferase and an alpha 2,3 sialyltransferase for increasing the number of α2,3,-α2,6-disialylgalactose N-glycans on a glycoprotein.

In one aspect, there is provided a pharmaceutical composition comprising a glycoprotein as defined herein.

In one embodiment, the pharmaceutical composition comprises a pharmaceutically acceptable carrier.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

Representative pharmaceutically acceptable carriers include any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient(s), its use in the pharmaceutical compositions is contemplated.

The pharmaceutical compositions may be in a variety of forms. These include, for example, liquid, semi-solid and solid dosage forms, such as liquid solutions (e.g., injectable and infusible solutions), dispersions or suspensions, liposomes and suppositories. The preferred form depends on the intended mode of administration and therapeutic application. Suitable pharmaceutical compositions can be administered intravenously, subcutaneously, intramuscularly, or via any mucosal surface, e.g., orally, sublingually, buccally, sublingually, nasally, rectally, vaginally or via pulmonary route. In specific embodiments, the compositions are in the form of injectable or infusible solutions. A preferred mode of administration is parenteral (e.g., intravenous, subcutaneous, intraperitoneal, intramuscular). In specific embodiments, the pharmaceutical composition is administered by intravenous infusion or injection. In other embodiments, the pharmaceutical composition is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives can also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin and/or by the maintenance of the required particle size. In specific embodiments, an agent of the present disclosure may be conjugated to a vehicle for cellular delivery. In these embodiments, the agent may be encapsulated in a suitable vehicle to either aid in the delivery of the agent to target cells, to increase the stability of the agent, or to minimize potential toxicity of the agent. As will be appreciated by a skilled artisan, a variety of vehicles are suitable for delivering an agent of the present disclosure. Non-limiting examples of suitable structured fluid delivery systems may include nanoparticles, liposomes, microemulsions, micelles, dendrimers and other phospholipid-containing systems. Methods of incorporating agents of the present disclosure into delivery vehicles are known in the art. Although various embodiments are presented below, it will be appreciate that other methods known in the art to incorporate a glycoprotein of the disclosure into a delivery vehicle are contemplated.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. A glycoprotein of the present disclosure can be administered on multiple occasions. Intervals between single dosages can be daily, weekly, monthly or yearly. Intervals can also be irregular as indicated by measuring blood levels of modified polypeptide or antigen in the patient. Alternatively, the glycoprotein can be administered as a sustained release formulation, in which case less frequent administration is required. Dosage and frequency vary depending on the half-life of the polypeptide in the patient.

It is especially advantageous to formulate compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutically acceptable carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

In one aspect, there is provided a glycoprotein as defined herein or a pharmaceutical composition as defined herein for use in therapy.

In one aspect, there is provided a method of treating a disease in a subject, the method comprising administering a glycoprotein as defined herein or a pharmaceutical composition as defined herein under conditions and for a sufficient time to treat the disease in the subject.

In one embodiment, there is provided the use of a glycoprotein as defined herein or a pharmaceutical composition as defined herein in the manufacture of a medicament for treating a disease in a subject.

The term “treating” as used herein may refer to (1) preventing or delaying the appearance of one or more symptoms of the disorder; (2) inhibiting the development of the disorder or one or more symptoms of the disorder; (3) relieving the disorder, i.e., causing regression of the disorder or at least one or more symptoms of the disorder; and/or (4) causing a decrease in the severity of one or more symptoms of the disorder.

The term “subject” as used throughout the specification is to be understood to mean a human or may be a domestic or companion animal. While it is particularly contemplated that the methods of the invention are for treatment of humans, they are also applicable to veterinary treatments, including treatment of companion animals such as dogs and cats, and domestic animals such as horses, cattle and sheep, or zoo animals such as primates, felids, canids, bovids, and ungulates.

The “subject” may include a person, a patient or individual, and may be of any age or gender.

The term “administering” refers to contacting, applying, injecting, transfusing or providing a composition of the present invention to a subject.

The pharmaceutical compositions of the invention may include an effective amount of the glycoprotein as defined herein. The effective amount may be a “therapeutically effective amount”. A “therapeutically effective amount” refers to an amount effective, at dosages and for periods of time necessary, to achieve the desired therapeutic result A therapeutically effective amount of the agent may vary according to factors such as the disease state, age, sex, and weight of the individual, and the ability of the agent to elicit a desired response in the individual. A therapeutically effective amount is also one in which any toxic or detrimental effects of the agent is outweighed by the therapeutically beneficial effects.

The disease may be, for example, AAT deficiency (or a condition associated with AAT deficiency), anaemia, cancer or diabetes.

In one embodiment, the glycoprotein is AAT and the disease is AAT deficiency or a condition associated with AAT deficiency.

In one embodiment, the glycoprotein is erythropoietin and the disease is anaemia.

Provided herein is a kit. The kit may comprise one or more reagents for increasing the number of α2,3,-α2,6-disialylgalactose on a glycoprotein in accordance with the present invention. For example, in one embodiment, the kit comprises an expression vector useful for recombinant expression of a recombinant glycoprotein. The kit may comprise a α2,6 sialyltransferase. The kit may contain a buffer for reacting the recombinant glycoprotein with the α2,6 sialyltransferase.

The kit may further comprise instructions. In other embodiments, the kit comprises separate compartments.

In one aspect, there is provided a kit for increasing sialylation of a glycoprotein, the kit comprising a column comprising an immobilized α2,6 sialyltransferase.

In one embodiment, the kit further comprises an immobilized α2,3 sialyltransferase and/or a β-1,4-galactosyltransferase.

In one embodiment, there is provided a kit comprising a column comprising an immobilized glycoprotein (such as alpha-1 antitrypsin (AAT)). The kit may allow an enzyme or enzyme mixture (such as a2,6 sialyltransferase and/or α2,3 sialyltransferase) to pass through and increase sialylation of the glycoprotein.

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

As used in this application, the singular form “a,” “an,” and “the” include plural references unless the context clearly dictates otherwise. For example, the term “an agent” includes a plurality of agents, including mixtures thereof.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

Certain embodiments of the invention will now be described with reference to the following examples which are intended for the purpose of illustration only and are not intended to limit the scope of the generality hereinbefore described.

### EXAMPLES

### Materials and Methods

### Experimental

(All materials were purchased from Sigma Aldrich unless otherwise stated)

rAAT Production

rAAT was produced in CHO-DG44 cells by the Animal Cell Technology (ACT) group at the Bioprocessing Technology Institute (BTI). rAAT was harvested after 10 days at 66% cell viability. An ÄKTA Purifier 10 (Amersham Pharmacia Biotech, United Kingdom) was used to purify the culture supernatant of rAAT. 30 mL of supernatant was diluted with 90 mL of deionised water to reduce the conductivity. The diluted supernatant was purified using a MonoQ 5/50 GL anion exchange column (GE Healthcare, Little Chalfont, United Kingdom) and eluted with 25% NaCl 50 mM Tris pH 7.5 buffer. The eluate was then concentrated to 5 mL using 10 kDa molecular weight cut off filtering units (Merck Millipore) and loaded onto the HiLoad 16/600 Superdex 200 prep grade size exclusion column (GE Healthcare). The protein of interest was eluted at 80 min and confirmed by Western blot with mouse AAT antibodies (48D2; Santa Cruz Biotechnology, Dallas Tex., USA) and anti-mouse IgG HRP conjugate (Promega, Madison Wis., USA). Commercial plasma AAT (Merck Millipore) was used as the positive control. The eluted fractions were combined, dried down, and reconstituted in water. Quantitation of protein concentration was done using the Pierce BCA protein assay kit (Thermo Scientific, Waltham Mass., USA) after desalting using 10 kDa molecular weight cut-off filter units (Merck Millipore) to avoid interference from the salts.

Incubation of rAAT with Glycosyltransferases

Incubation of rAAT with a2.6 sialyltransferase from Photobacterium damselae

50 μg of rAAT was incubated with, 100 mM Tris HCl, 2 mM Cytidine monophosphate N-acetylneuraminic acid (CMP-Neu5Ac), and 25 μg of a2,6 sialyltransferase from Photobacterium damselae (Sigma Aldrich) in a total volume of 100 μL. The samples were incubated for 16 hrs (overnight) using a heating block set at 37° C. with 300 RPM agitation. The reaction was prepared in triplicate.

Incubation of rAAT with a2.6 Sialyltransferase from Photobacterium damselae and D1,4-Galactosyltransferase from Bovine Taurus Milk

50 μg of rAAT was incubated with, 100 mM Tris HCl, 2 mM Uridine-diphospho-galactose (UDP-Gal), 2 mM CMP-Neu5Ac, 5 mM MnCl₂, 20 μg of 01,4-galactosyltransferase from Bovine Taurus milk (Sigma Aldrich), and 25 μg of a2,6 sialyltransferase from Photobacterium damselae (Sigma Alrich) in a total volume of 100 μL. The samples were incubated for 16 hrs (overnight) using a heating block set at 37° C. with 300 RPM agitation. The reaction was prepared in triplicate.

Incubation of rAAT with a2.6 Sialyltransferase from Photobacterium damselae, β1,4-Galactosyltransferase from Bovine Taurus Milk and α2.3 Sialyltransferase from Pasteurella multocida

50 μg of rAAT was incubated with, 100 mM Tris HCl, 2 mM UDP-Gal, 2 mM CMP-Neu5Ac, 5 mM MnCl₂, 20 μg of 01,4-galactosyltransferase from Bovine Taurus milk (Sigma Aldrich), and α2,3 sialyltransferase from Pasteurella multocida (Sigma Aldrich) in a total volume of 100 μL. The samples were incubated for 16 hrs (overnight) using a heating block set at 37° C. with 300 RPM agitation. Following overnight incubation an additional 2 mM CMP-Neu5Ac and 25 μg of
a2,6 sialyltransferase from Photobacterium damselae was added to the reaction and incubated for a further 4 hrs. The reaction was prepared in triplicate.

N.B. All Incubations were Carried Out in Triplicate

Proteolytic Digestion

Following enzymatic incubations the rAAT was denatured in 4M urea in a final volume of 200 μL. 30 μL of 10 mM Dithiothreitol (DTT) in 4 M urea was added to the sample and incubated for 30 mins at 56° C. The sample was then transferred to a 10 kDa molecular weight cut-off filter units (PALL) and centrifuged to remove DTT (14000 rcf 10 mins). Fifty microliters (50 μL) iodoacetic acid (15 mM in 0.1M Tris, pH 8.0) was then added to the sample on the membrane and incubated at room temperature in the dark for 30 minutes. The iodoacetic acid was removed by centrifuging for 10 minutes. The sample was then washed 3 times with 300 μL 50 mM ammonium bi-carbonate. 50 μL of ammonium bicarbonate was added followed by trypsin to the sample in a 1:20 trypsin:protein ratio (5 μL of 20 μg/μL Sequencing Grade Trypsin Promega) and left overnight at 37° C. After incubation the sample was centrifuged with the filtrate being collected. The membrane was washed with 100 μL 50 mM ammonium bicarbonate then 100 IL water with filtrate being collected. The filtrate was then evaporated to dryness and reconstituted in 100 μL 0.1% formic. 1 μL of diluted sample was aliquoted and diluted ×10 further in 0.1% formic acid to give an approximate concentration of 50 ng/μL of rAAT. This was then analysed using an LC-MS orbitrap instrument.

LC-MS/MS of Tryptic Digested Glycopeptides

Fifty nanograms (50 ng) of tryptic digested rAAT was injected onto a Thermo Scientific LC-MS/MS using the following setup.

A PepMap RSLC C18 nanoflow Easy Spray column (2 um diameter×10 nm beads 15 cm length) (Thermo fisher Scientific,) at 40° C. with a flow rate of 300 nL/min was used for glycopeptide separation. The mobile phase A was 0.1% formic acid in water and mobile phase B 0.1% formic acid in acetonitrile. The analytical gradient lasted for 110 min where after 10 min balancing time, solvent B rose from 4% to 50% over 105 mins. Solvent B was increased to 95% in 5 min and was held for 7 min and then returned to 4% B in 5 min and was held for 21 min. The LC was coupled to an Orbitrap Fusion Tribrid mass spectrometer (Thermo Fisher Scientific) operated in positive ion mode. HCD MS/MS (HCD energy 25%, 5 s duty cycle) was performed on precursors with charge 2-8, a dynamic exclusion of 12 seconds and isolation window of m/z=±1.6 with peptide monoisotopic peak detection. The fragments were detected by an Orbitrap detector. A full scan range of m/z 400 to 2600 was used.

LC-MS/MS Glycopeptide Data Analysis

Byonic Settings

The search peptide engine byonic was used for glycopeptide identification using the following settings. 2 missed cleavages were allowed with precursor mass tolerance of 25 ppm, and fragment mass tolerance of 0.5 Da. Modifications included cysteine carboxymethylation (+58.005 Da) and methionine oxidation (+15.995 Da). N-Glycan library was used from data obtained from released glycomics and previous studies on CHO glycans. This list was then gradually refined based on initial results and our own studies.

OPEN-MS Knime Settings

The raw data underwent an OPEN-MS, Knime workflow in order for quantitation of glycopeptides to take place. Briefly, raw files were converted to *.mzML using MSConverter.

The converted files were passed through a knime workflow consisting of peak picking (PeakpickerHiRes), feature finding (FeatureFinderHm) and decharging. The result produced consensus files containing decharged features with mass, intensities and retention times.

Matching of Byonic & Knime Output for Data Analysis

The output from the byonic and knime workflow were matched together based on matching mass and retention times between the two sets of data. Briefly the glycopeptides identified by the byonic software were matched with the intensity of the features observed from the knime workflow output with similar m/z and retention time in each set of data. This was eventually automated using an in house python script Following the matching the relative abundance of the glycans was determined based on relative intensity. A number of properties of the glycan data such as the levels of sialylation were also calculated. All error was determined by standard error of the mean. This was also eventually automated using a separate in house python script.

Synthesis and Analysis of Disialyllactose

Neu5Ac-a2,3(Neu5Ac-a2,6)Galb1-4Glc (DSLac) was synthesised by incubating 0.01 mM of a2,3 sialyllactose with 100 mM Tris, 2 mM CMP-Neu5Ac and 12.5 μg α2,6 sialyltransferase from Photobacterium damselea (Sigma Aldrich). The enzyme was removed using a 10 kDa molecular weight cut-off filter units (Vivaspin). The sample was then dried down, and suspended in a 50 μL solution of ethanol, 0.25M 1-Ethyl-3-(3-dimethylaminopropyl)carbodiimide (EDC) and 0.25 M Hydroxybenzotriazole (HOBt) for 1 hr at 37° C. to allow esterificastion/lactonisation reactions of the sialic acid groups as described previously. The esterification reaction was repeated for Neu5Ac-a2,3-Neu5Ac-a2,3-Galb1-4Glc (PSLac). Following the reaction 1 μL of the sample was mixed directly onto a MALDI plate with 1 μL of a 20 mg/mL matrix solution of 3,4 Diaminobenzophenone in 50:50 ACN:H₂O solution. A AB SCIEX TOF/TOF™ 5800 System was used in positive ion mode with settings described below.

MALDI-TOF-MS Settings

MALDI-TOF-MS spectra of were obtained in positive ion reflector mode, the laser intensity was varied between 3500-6000 with a pulse rate of 400 Hz until a desirable spectrum was observed. 200 hundred shots/per sub spectrum and 2000 shots per spectrum were used. Continuous stage motion was used with a velocity of 600 μm/second. 2-5 spectra were accumulated until a desirable spectrum was obtained.

MALDI-TOF-MS/MS Settings

The desired precursor mass was identified and MALDI-TOF-MS/MS spectra were obtained in positive ion mode with Argon as the CID gas for fragmentation. The precursor mass window was set at a resolution 200 (FWHM). The laser intensity was varied between 4500-6000 with a pulse rate of 1000 Hz until a desirable spectrum was observed. 200 hundred shots/per sub spectrum and 2000 shots per spectrum were used to generate a spectrum. The stage was moved after every sub-spectrum. 5-10 spectra were accumulated until a desirable accumulated spectrum was obtained.

Ethyl-Esterification of rAAT Glycopeptides.

A 50 μL aliquot of a2,6 PTB treated rAAT tryptic digest (25 μg) was taken and evaporated to dryness. Once dry the peptide mixture was resuspended in 20 μL of ethanol, 0.25M 1-Ethyl-3-(3-dimethylaminopropyl) carbodiimide (EDC) and 0.25M hydroxybenzotriazole (HOBt) and left at room temperature for 1 hr with light shaking. Following incubation the sample was diluted with 20 μL acetonitrile and stored at −20° C. Prior to analysis the solution underwent a glycopeptide HILIC enrichment. Briefly cotton was placed into a 20 μL pipette tip and using a syringe needle (0.2-0.5 mm of cotton in the tip). The cotton filled pipette tips were washed by pipetting ten times 20 μL water, followed by equilibration with 10 times 20 μL 85% acetonitrile. The samples were loaded by pipetting the sample solution 15-25 times up and down. The cotton tips were then washed three times with 20 μL of 0.1% TFA in 85% acetonitrile and five times with 20 μL of 85% acetonitrile. The sample was eluted with 10 μL water. 5 μL of the eluted sample following cotton HILIC clean-up was diluted in 15 μL of 0.1% formic acid and analysed on an LC-MS Orbitrap Instrument (see setting below). (N.B. It is worth noting that the sample seemed to degrade with evidence of underivatised sialic acid being observed after one day left in a sample vial so analysis should be conducted immediately.)

LC-MS/MS settings for Ethylated Glycopeptides

A PepMap RSLC C18 nanoflow Easy Spray column (2 um diameter×10 nm beads 15 cm length) (Thermo fisher Scientific,) at 40° C. with a flow rate of 300 nL/min was used for glycopeptide separation. The mobile phase A was 0.1% formic acid in water and mobile phase B 0.1% formic acid in acetonitrile. The analytical gradient lasted for 52.5 minutes where after 2.5 minutes balancing time, solvent B rose from 4% to 50% over 37.5 minutes. Solvent B was increased to 95% in 5 min and was held for 5 min and then returned to 4% B in 5 minutes and was held for 5 minutes. The LC was coupled to an Orbitrap Fusion Tribrid mass spectrometer (Thermo Fisher Scientific) operated in positive ion mode. HCD MS/MS (HCD energy 25%, 5 s duty cycle) was done on precursors with charge 2-8, a dynamic exclusion of 12 seconds and isolation window of m/z=f 1.6 with peptide monoisotopic peak detection. The fragments were detected by an Orbitrap detector. A scan range of m/z 1000 to 1600 was used.

Permethylation of Released rAAT Glycans

A 50 μL aliquot of a2,6 PTB treated rAAT tryptic digest (25 μg) was evaporated to dryness. Once dry the peptide mixture was resuspended in 96 μL 50 mM ammonium bicarbonate and 2 U (4 μL of 500 U/mL New England Biolabs) recombinant PNGaseF was added. The sample was incubated overnight (17 hrs) at 37° C. Following incubation the released N-Glycans were purified from the mixture using a waters Sep-Pak Vac 200 mg C18 solid phase extraction cartridge (Waters). Briefly the cartridge was prepared by passing through 2 columns of methanol followed by 2 columns of water 2 columns of acetonitrile and another 2 columns of water. The released glycan solution was acidified by addition of one drop of acetic acid (glass pipette) before being loaded onto the column. The peptides were allowed to bind to the cartridge and the filtrate containing the released glycans collected in the flow through in a glass vial, the column was washed with 1-2 mL water to ensure all the glycans were eluted from the column. The elutant was then dried under N2. Peptides were also then eluted from the column with 50% acetonitrile and collected separately. Once dried the glycans were permethylated as outlined in the literature, briefly a 1.5 mL slurry of 5 crushed NaOH pellets in DMSO was transferred to the glass vial containing the released N-Glycans and briefly shaken. Then 900 μL iodomethane was added to the mixture. The solution was shaken for 45-60 minutes to ensure complete permethylation. Following shaking 1 mL of water was added to quench the reaction, the sample was then vortexed. Then 2 mL chloroform was added to the sample and vortexed. The sample was then spun at 1000 g in a centrifuge to allow for separation of organic and aqueous layers. The top aqueous layer was removed, then an additional 2 mL of water was added to the sample. The sample was then washed a further 5-7 times with water (repeating the previous washing step described). Once the final aqueous layer had been removed the organic layer was dried down under N2. The permethylated N-glycans were then resuspended in 20 μL of methanol. 2 μL aliquot was then taken and mixed with 2 μL of 20 mg/mL DHB in 50:50 ACN:H₂O. 2 μL spots were then spotted onto a MALDI target the plate. An AB SCIEX TOF/TOF™ 5800 MALDI-TOF-MS/MS was used for the analysis and settings are described below.

MALDI-TOF-MS Settings

MALDI-TOF-MS spectra of permethylated N-glycans were obtained in positive ion reflector mode, the laser intensity was varied between 3500-6000 with a pulse rate of 400 Hz until a desirable spectrum was observed. 200 hundred shots/per sub spectrum and 2000 shots per spectrum were used. Continuous stage motion was used with a velocity of 600 μm/second. 2-5 spectra were accumulated until a desirable spectrum was obtained.

MALDI-TOF-MS/MS Settings

The desired precursor mass was identified and MALDI-TOF-MS/MS spectra were obtained in positive ion mode with Argon as the CID gas for fragmentation. The precursor mass window was set at a resolution 200 (FWHM). The laser intensity was varied between 4500-6000 with a pulse rate of 1000 Hz until a desirable spectrum was observed. 100 hundred shots/per sub spectrum and 10000 shots per spectrum were used to generate a spectrum. The stage was moved after every sub-spectrum. 5-10 spectra were accumulated until a desirable accumulated spectrum was obtained.

Neuraminidase Activity Tests

Twenty five (25 μL) of tryptic digested a2,6PTB treated rAAT (12.5 μg) was evaporated to dryness and resuspended in 15 μL of water. Two (2 μL) of glycobuffer 1 (NEB) was added along with 3 μL (60 U) ABS (NEB). The sample was incubated overnight at 37° C. Following incubation the reaction was stopped by passing the sample through a 10 kDa membrane filter (PALL). The sample was then evaporated to dryness and then resuspended in 25 μL of 0.1% FA. The sample was analysed using the LC-MS/MS Orbitrap Instrument under the same settings described for the ethylated glycopeptides previously.

rAAT Activity Analysis

Purification of Remodeled rAAT

Triplicate incubations were run as described above (c.f. Incubation of rAAT with α2,6 sialyltransferase from Photobacterium damselae, β1,4-galactosyltransferase from Bovine Taurus milk and α2,3 sialyltransferase from Pasteurella multocida). Following the incubation, two of the replicate incubations were combined and concentrated in a 10 kDa ultrafiltration device (Amicron) and washed with 100 μL of water. (The final replicate underwent glycopeptide analysis to ensure the in vitro glycan remodelling had been successful.) The concentrated enzyme treated rAAT was removed from the filtration device by pipetting up and down with 100 μL of water and slight shaking. The filter unit was then tipped upside down and centrifuged at 1000 g for 2 minutes. The recovered protein was then diluted 3 times in 20 mM Tris, 150 mM NaCl, pH 7.5. A centrifugal column unit (Pierce™ Spin Columns-Snap Cap Thermo Fisher) was then prepared which contained 200 μL of Alpha-1-Antitrypsin Select (GE Healthcare Life Sciences) that had been washed in 20 mM Tris, 150 mM NaCl, pH 7.5. The enzyme treated rAAT protein was then loaded onto the column (under gravity), the filtrate was reapplied to the column three times to ensure the majority of the enzyme treated rAAT bound to the resin. The resin was then washed with 20 mM Tris, 150 mM NaCl, pH 7.5, with 4-5 column volumes making sure that the resin did not go dry. Once the resin wash washed, 20 mM Tris, 2 M MgCl₂, pH 7.5 solution was passed through the column to elute the enzyme treated rAAT from the resin. 4-5 column volumes were passed over the column and all filtrate being collected. To ensure all of the enzyme treated rAAT was unbound from the resin, on the final elution the resin was disturbed by pitpetting up and down with the 20 mM Tris, 2 M MgCl₂, pH 7.5 before being allowed to settle and the filtrate being collected. The filtrate was collected and ran on a nandrop to ensure protein was present in the elution. Once confirmed, the enzyme treated rAAT in the elutant was concentrated on a 10 kDa ultrafiltration device (Amicron) washing with 100 μL of water before the purified enzyme treated rAAT was removed from the filtration device by pipetting up and down with 100 μL of water and slight shaking. The filter unit was then tipped upside down and centrifuged at 1000 g for 2 minutes and the purified enzyme treated rAAT collected in a final volume of around 100-150 μL.

ELISA Titre Analysis

To determine an accurate concentration of the enzyme treated rAAT following purification the enzyme treated rAAT was analysed using Human alpha-1-antitrypsin ELISA Quantitation kit (GenWay Biotech, Inc., USA) according to manufacturer's instructions, but with a commercially available plasma-derived AAT (Abcam, United Kingdom, Cat. No. ab91136) to generate the standard curve. The concentration observed during the analysis was then used for the elastase activity assay.

Neutrophil Elastase rAAT Activity Assay Remodeled rAAT activity was measured by incubating samples with excess porcine pancreatic elastase (Merck, Germany, Cat. No. 324682) for 30 min, and assaying the remaining elastase activity by the kinetic hydrolysis of SAPNA (Sigma-Aldrich Corporation, USA, Cat. No. S4760), measured at 410 nm. Sample remodeled rAAT activities were compared to a standard curve generated by loading different amounts of plasma-derived AAT (Abcam, United Kingdom, Cat. No. ab91136) to determine the amount of active AAT in the samples as compared to the plasma-derived AAT. The amount of active rAAT was then divided by the amount of rAAT loaded into the activity assay to determine the relative percentage active rAAT in the sample, with the activity of the plasma derived AAT set at 100%.

### Example 1

The primary premise behind the use of the α2,6 sialyltransferase photobacterium for in vitro glycan remodeling of biotherapeutics is to exploit its unique activity to add a2,6 silaic acids to already α2,3 sialylated glycans of glycoproteins, and to produce disialylgalactose N-glycan structures on a glycoprotein. This new approach can enhance the in vitro sialylation of biotheraputics.

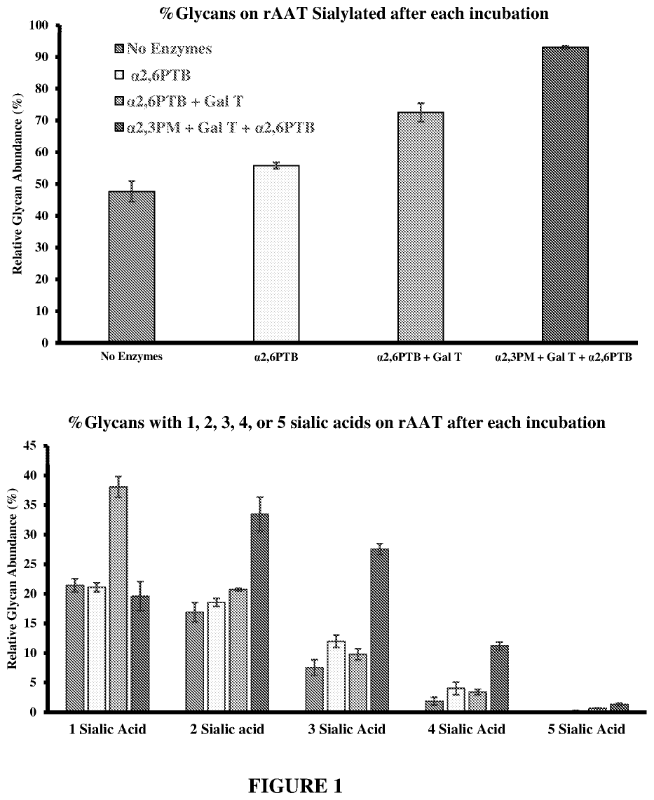

To explore this further, bacterial a2,6 sialyltransferase from Photobacterium damselae (a2,6 PTB) (Sigma Aldrich) was used to remodel the N-Glycans of recombinantly Chinese Hamster Ovary (CHO) produced AAT (rAAT). Subsequent analysis of the rAAT sialylation by glycopeptide analysis using LC-MS/MS following rAAT tryptic digestion showed increases in relative sialylation was small with only an 8% increase in the number of glycans sialylated in rAAT following incubation with a2,6PTB (FIG. 1a). The SA count takes into account how highly sialylated glycans are i.e. mono, di, tri etc. An unexpected rise in average SA count to 3.3 was seen, despite the small increase in the number of glycans that were newly sialylated (Table 1). An increase of 7% in glycans that were tri and tetra sialylated (FIG. 1b) suggested that the α2,6 PTB has sialyation activity towards already sialylated glycans leading to a surprisingly large increase in average SA count considering the small increase in newly sialylated glycans.

The rAAT has 44% of glycan species with terminal N-Acetyl-Hexosamine (HexNAc) (Table 2-3). Therefore a second glycosyltransferase β-1,4-Galactosyltransferase from Bovine Taurus (GalT) (Sigma Aldrich) was introduced to increase the galactosylation of the rAAT N-Glycans and thus the substrate for sialylation. Incubation of the rAAT with a combination of a2,6 PTB and GalT gave a 25% increase in the number of glycans sialylated and average SA count of 3.8 (FIG. 1a & Table 1). Relatively there was a larger proportion of mono sialylated glycans (38%) compared with the untreated rAAT (21%) and the α2,6PTB treated rAAT (21%), with only small increases in the bi (4%) and tri sialylated (2%) glycans (FIG. 1b). The large increase in the monosialylated glycans meant the average SA count of 3.8 was much higher than that observed for the α2,6 PTB reaction with no GalT (Table 1).

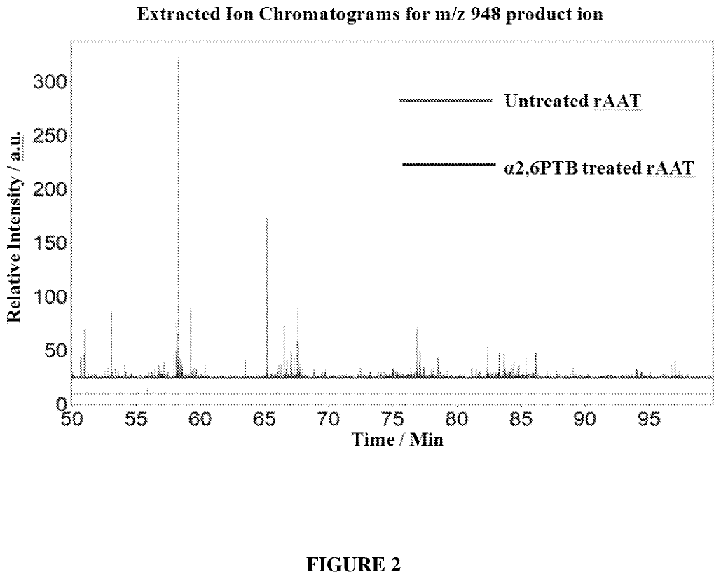

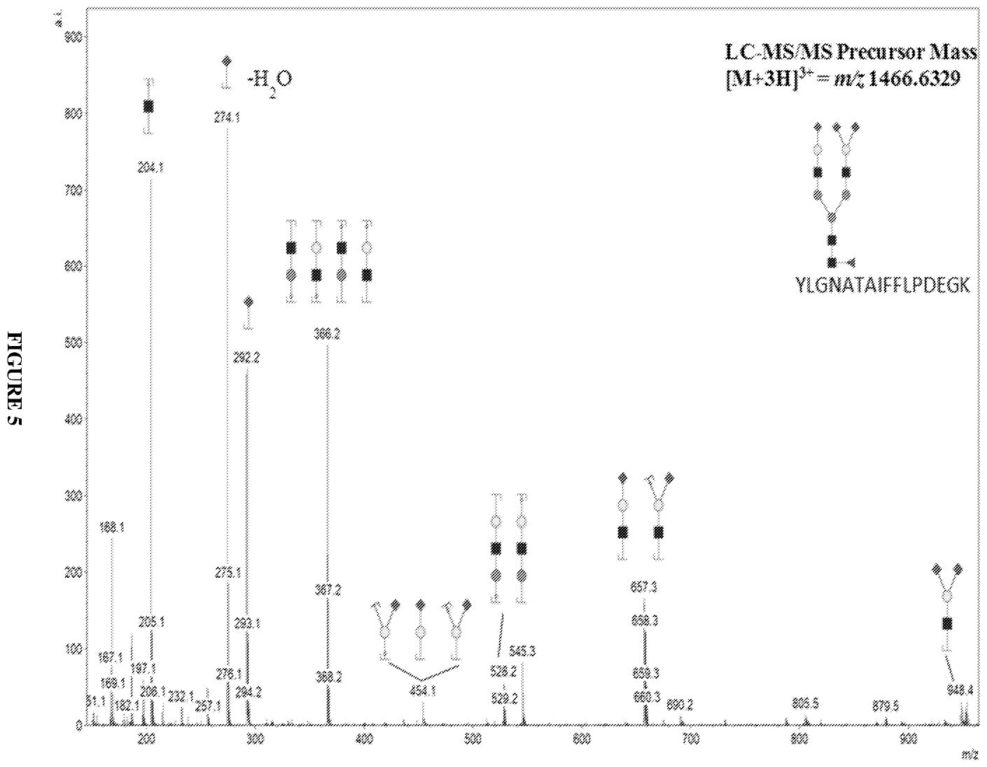

To try and better understand a2,6 PTB activity towards already sialylated glycans, the inventors further investigated the LC-MS/MS data. The inventors discovered that a number of glycans were hypersialylated (contained two Neu5Ac on a single glycan antennae). Inspection of the MS/MS for those hypersialylated glycopeptides showed HexNAc₁Hexose₁NeusAc₂B₃ m/z 948 product ion fragments in the MS/MS (FIG. 5). No significant m/z 948 product ions were observed in the extracted ion chromatogram of any rAAT glycopeptides prior to α2,6 PTB incubation, confirming the observed hypersialylation is the result of the α2,6PTB activity (FIG. 2).

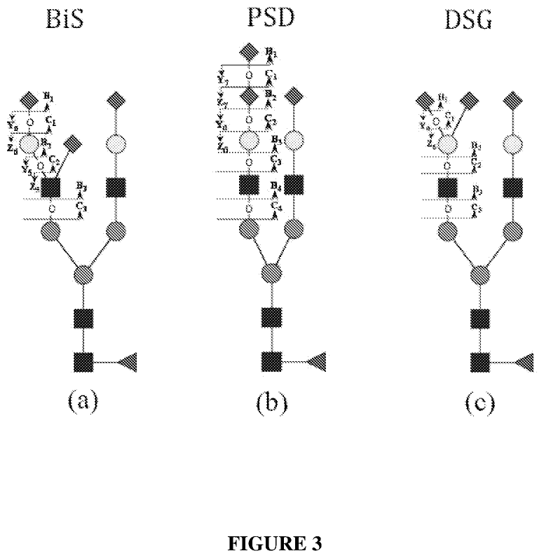

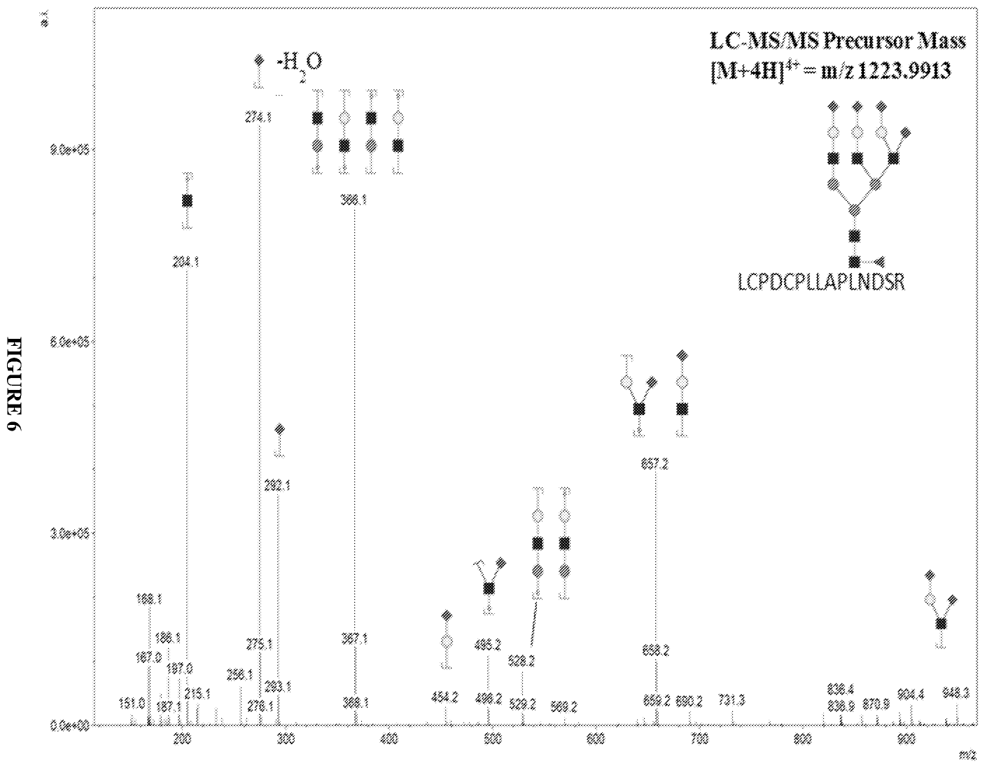

The nature of the hypersialylated N-Glycan species is of some interest. Similar structures have been observed in N-Glycans of Fetuin (Bovine Taurus) due to a bisecting sialic acid on N-Acetyl-Glucosamine (GlcNAc) (FIG. 3a). However, a2,6 PTB has no sialylation activity for GlcNAc substrate18 and no HexNAc₁Neu₅Ac₁ C₃Z₅/B₃Y₅ product ions (m/z 495) were observed in the MS/MS that typically is seen for bi-secting GlcNAc sialylation (BiS) or any alternative HexNAc₁Neu5Ac₁ sialylation (FIGS. 5 and 6). The hypersialylated glycans on rAAT are likely not the result of BiS. Hypersialylation could also result from polysialic acid disialylation (PSD) (Neu5Ac-α2,8-Neu5Ac-α2,3-Gal) sometimes seen on N-Glycans (FIG. 3b). In our studies, no Neu5Ac₂ fragments were observed in the MS/MS of the hypersiaylalted glycopeptides. In addition α2,6 PTB is not known to have any PSD activity. Instead the most likely source of the hypersialylated glycans on the rAAT is a single galactose residue that has been sialylated at both the 3 and 6 position of the terminal galactose, producing terminal α2,3-α2,6-diasialylgalactose-sialylation (Neu5Ac-a2,3(Neu5Ac-a2,6)Gal) (DSG) (FIG. 3c). α2,6 PTB can sialylate α2,3-sialyllactose to produce Neu5Ac-a2,3(Neu5Ac-a2,6)Galb1-4Glc (DSLac), structures. As rAAT was produced in CHO cells all sialic acids present in the rAAT are α2,3 sialylated. Therefore, a large amount of α2,3 sialylated galactose acceptor substrate is available for the α2,6PTB to add additional α2,6 sialic acids on the rAAT.

Standard LC-MS/MS of the hypersialylated glycopeptides of rAAT was not successful in confirming the DSG sialylation. The absence of the characteristic product ions that are seen for the PSD and BiS were the only indicator that the α2,6 PTB treated rAAT had DSG sialylation. This could easily have been overlooked especially as no Hexose₁Neu5Ac₂ product ions were observed, likely due to the liable nature of the Neu5Ac. Therefore, derivatization of the Neu5Ac was required to observe characteristic fragments and allow for complete characterisation of the N-Glycans by MS to confirm DSG glycan structures.

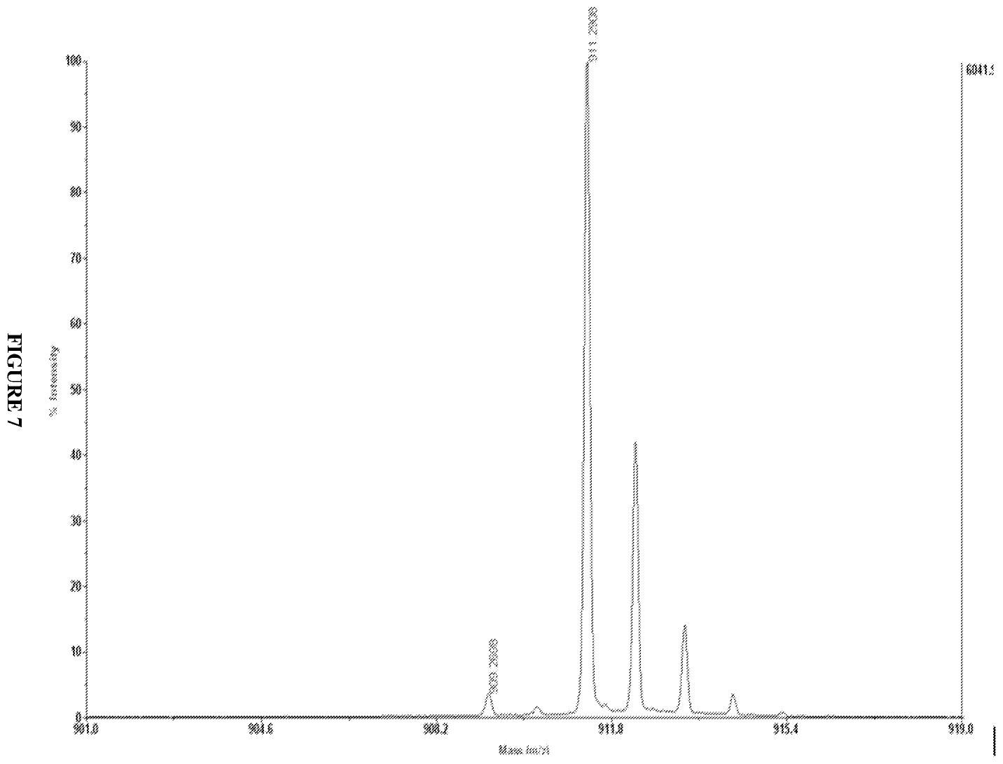

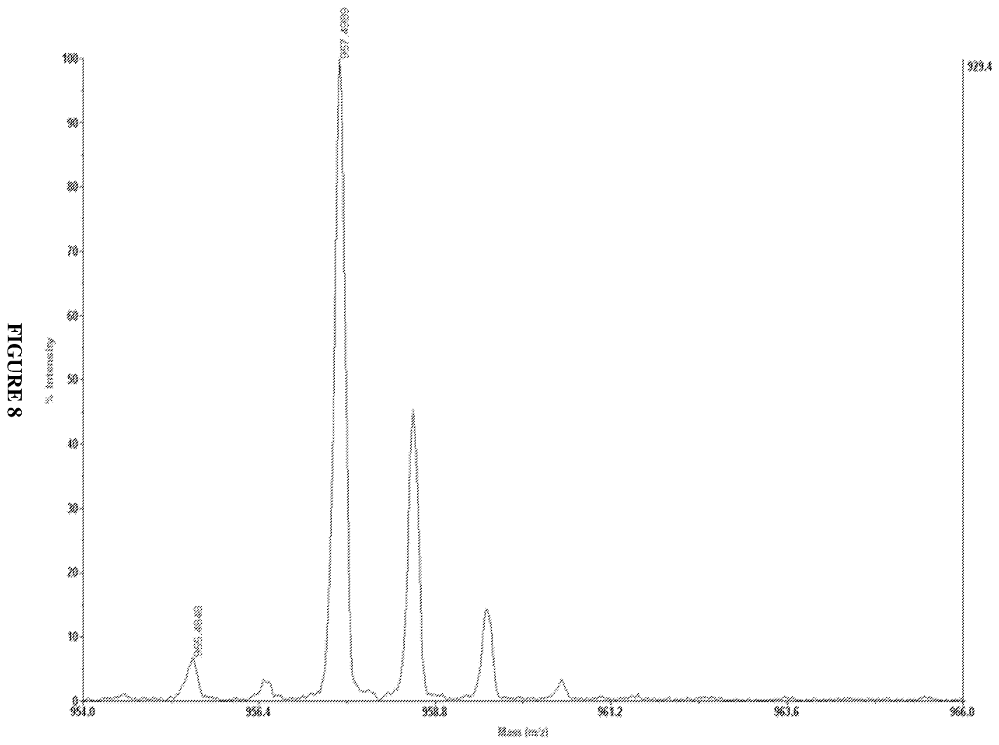

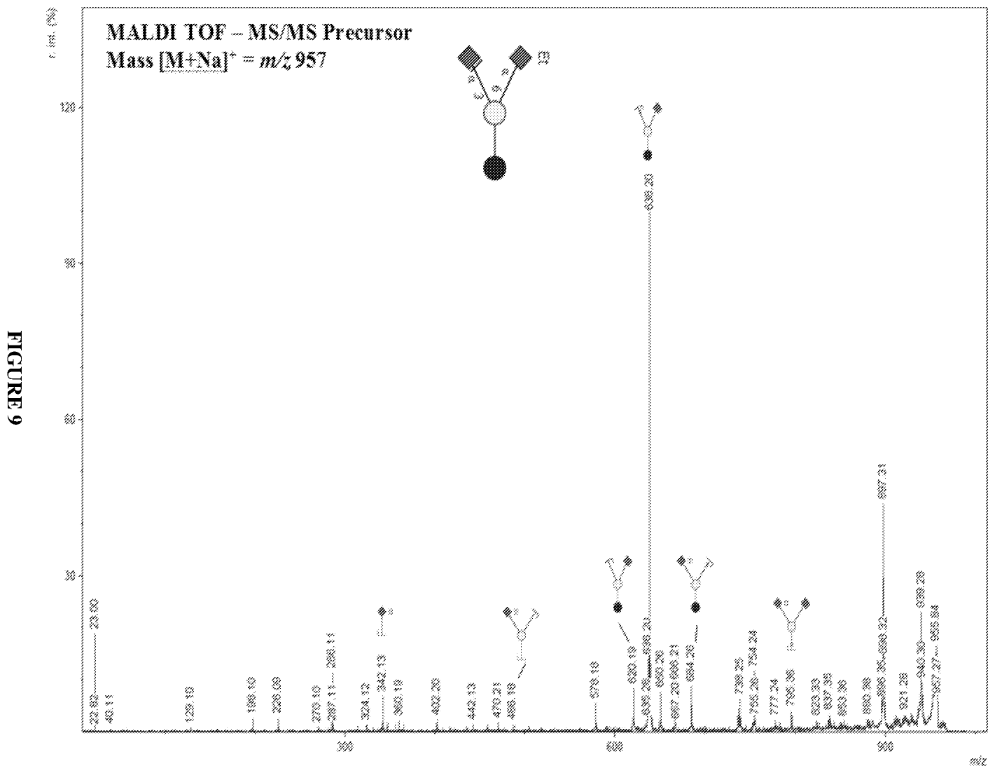

The inventors performed ethyl-esterification of the sialic acids to further investigate the nature of sialylation. Ethyl-esterification will lactonize α2,3/a2,8 sialic acids with subsequent loss of water (m/z −18), while a2,6 sialic acids gain an ethyl group (m/z +28). This allows for differentiation between the sialic acids isomers in the MS. As proof of concept we ethyl-esterified a synthesised standard of DSLac and an Neu5Ac-α2,8-Neu5Ac-α2,3-Galb1-4Glc (PSLac) standard and analysed them by MALDI-TOF-MS/MS. The results conclusively showed that the two species can be differentiated. The PSLac showed both sialic acids form lactones following the ethyl-esterification reaction resulting in the loss of two water molecules (m/z −36) and a molecular species [M+Na]+=m/z 911 in the MS (FIG. 7). In contrast DSLac had a molecular species [M+Na]+=m/z 957 (FIG. 8). This corresponds to one sialic acid being a lactone (m/z −18) following the reaction while another sialic acid has been ethyl-esterified (m/z+28) resulting in an overall mass increase of m/z+10 and a [M+Na]+=m/z 957. MS/MS of the ethyl-esterified DSLac confirmed both sialic acid were on a single hexose producing a m/z 795 Hexose₁Neu5Ac₂ C₂ product ion fragment (FIG. 9). The presence of both lactonized (m/z 638) and ethylated (m/z 684) Y₃ Hexose₁Neu5Ac₁ product ions also ruled out any unlikely Neu5Ac-α2,8-Neu5Ac-α2,6 isomers. This showed we can distinguish between PSLac and DSLac in the MS and eliminate the BiS sialylation and other potential isomers using MS/MS.

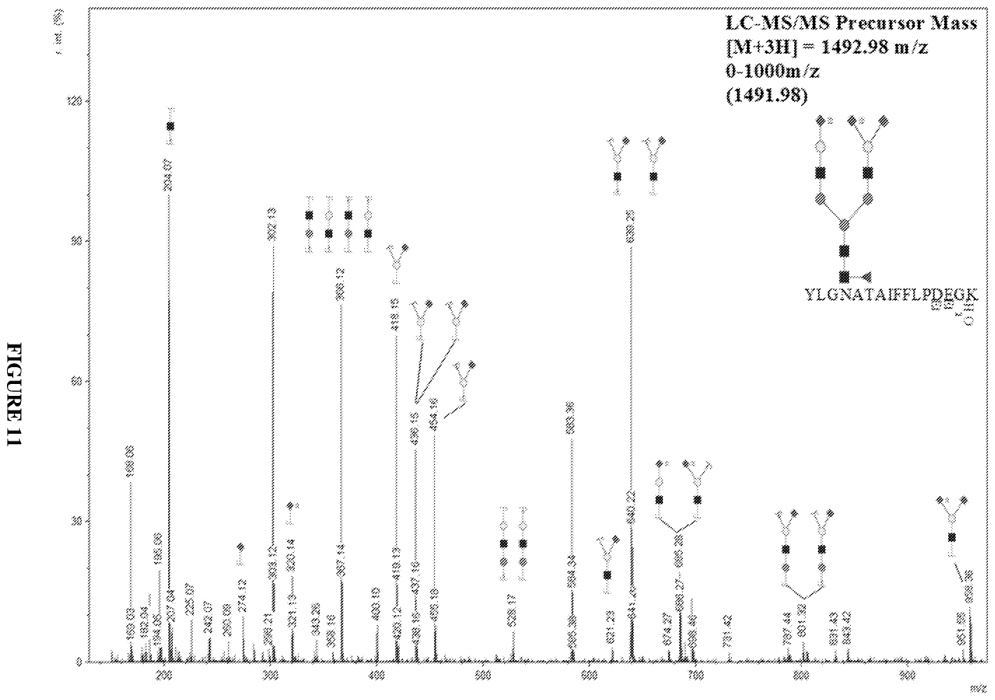

As ethyl esterification successfully confirmed the presence of the doubly sialylated galactose on DSLac by MALDI-TOF-MS/MS, the inventors tried a similar approach on the hypersialylated tryptic glycopeptides of α2,6 PTB treated rAAT using LC-MS/MS for analysis. The ethylated hypersialylated glycopeptides were identified in the LC-MS/MS and the MS/MS produced a unique product ion at m/z 958 (FIG. 11). This product ion is consistent with a HexNAc₁Hexose₁Neu5Ac₂ B₃ product ion fragment with a m/z+10 shift compared with the HexNAc₁Hexose₁Neu5Ac₂ B₃ (m z 948) product ion fragment prior to the ethylation reaction. This suggested that the hypersialylated structures observed on the rAAT following α2,6 PTB treatment is from DSG sialylation. However, no definitive Hexose₁Neu5Ac₂ DSG C₂ product ions were observed. Lack of the expected Hexose₁Neu5Ac₂ product ions meant that DSG could not be fully confirmed to be on the rAAT. Therefore the inventors decided to permethylate the released glycans of α2,6 PTB treated rAAT to stabilize the N-Glycans enough to observe the Hexose₁Neu5Ac₂ product ions.

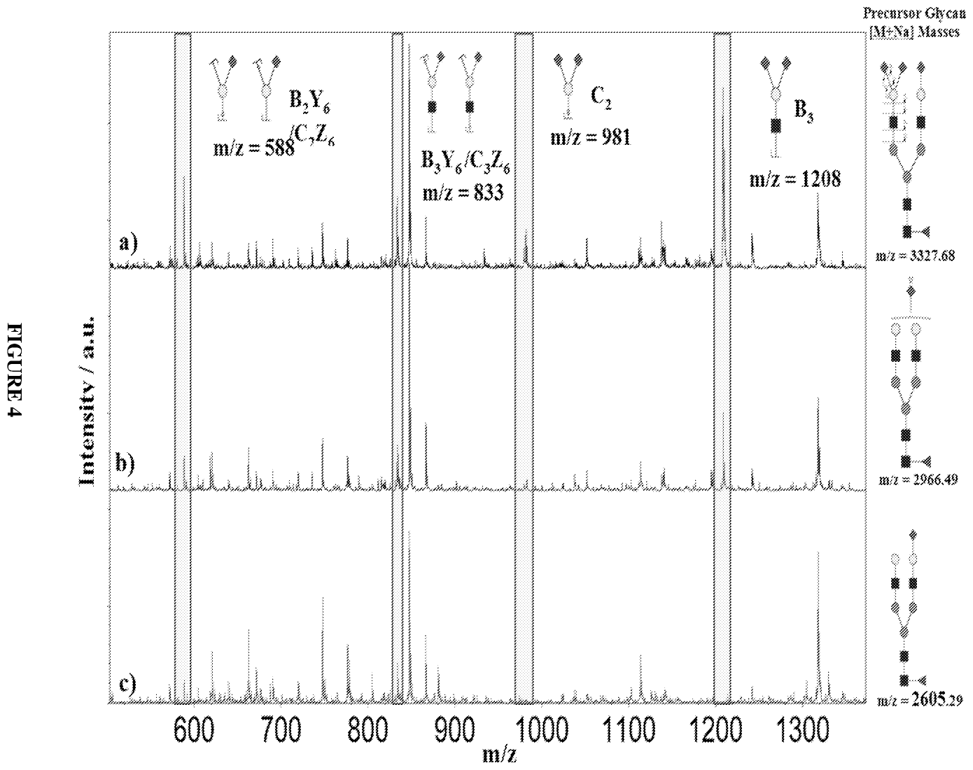

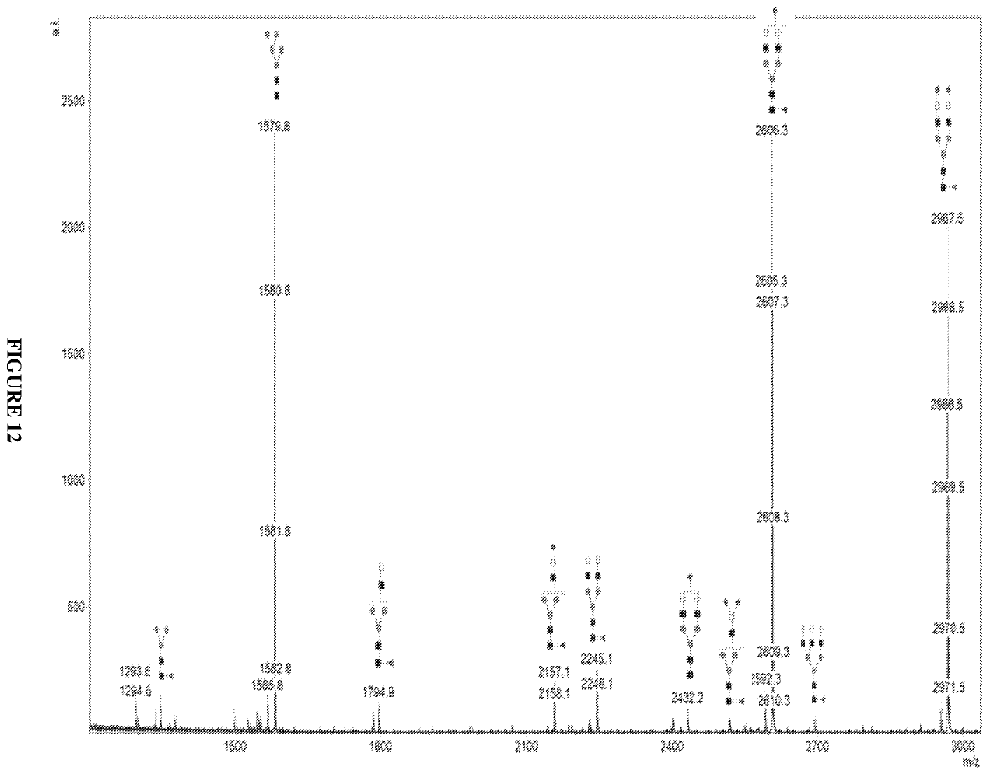

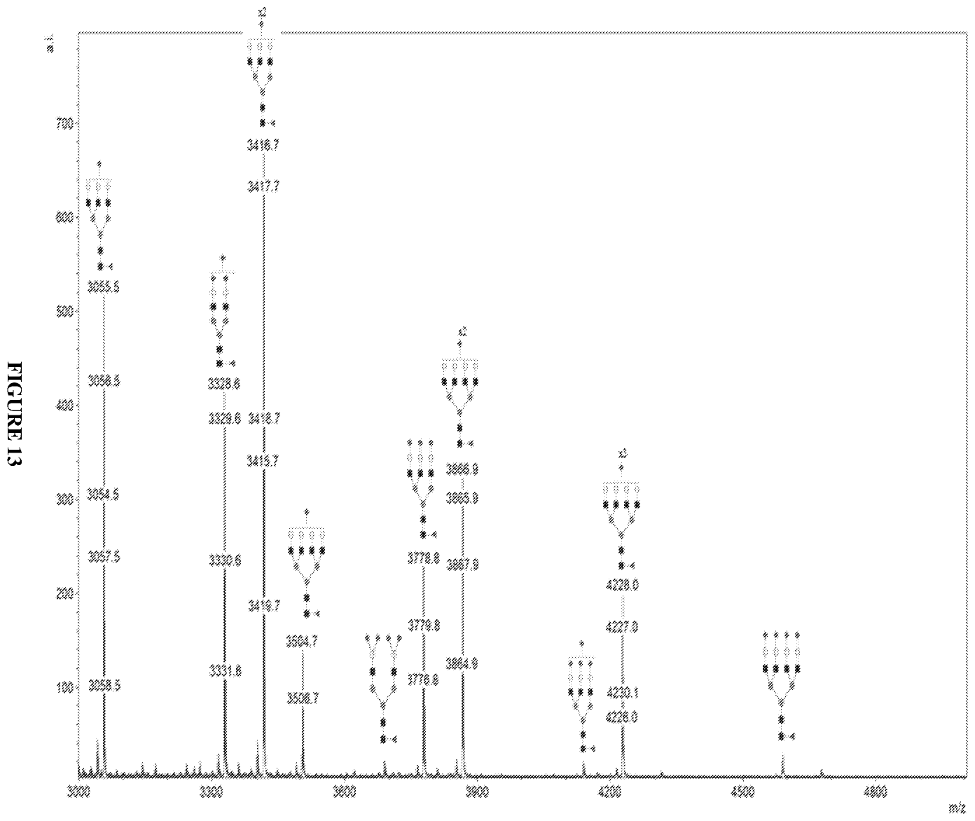

N-Glycans were released from a trypsin digest of α2,6 PTB treated rAAT, permethylated and analysed on a MALDI-TOFMS/MS. A number of molecular species consistent with hypersialylated N-Glycans were observed (Table 6, FIG. 12-13). The inventors took the HexNAc₄Hexose₅Neu5Ac₃ permethylated glycan ([M+Na]+=m/z 3327.61]) and performed MALDI-TOF-MS/MS to confirm DSG sialylation. The MS/MS of the HexNAc₄Hexose₅Neu5Ac₃ produced unique fragments when compared to other sialylated species in the sample (FIG. 4). In brief a unique C2 Hexose₁Neu5Ac₂ product ion at m/z 981 was observed. A significant increase in intensity of the product ions at m/z 588 due to B₂Y₆/C₂Z₆ Hexose₁Neu5Ac₁ product ions and at m/z 833 due to HexNAc₁Hexose₁Neu5Ac₁ B₃Y₆/C₃Z₆ product ions. An intense B₃ HexNAc₁Hexose₁NeuAc₂ product ion was observed for HexNAc₄Hexose₅Neu5Ac₃ at m/z 1208 but was not observed for the singly sialylated glycan HexNAc₄Hexose₅Neu5Ac₁ (FIG. 4a & 4c). Interestingly the B₃ product ion was observed in the MS/MS of the disailylated glycan HexNAc₄Hexose₅Neu5Ac₂ (FIG. 4b). This suggests that a small amount of the disalylated glycan species following α2,6 PTB treatment are DSG isomers. The permethylated product ion fragments in combination with the ethyl esterification results, and understanding of the enzyme activity, confirmed that the hypersialylation on the rAAT is the result of DSG sialylation activity of α2,6 PTB.

Having confirmed the DSG on rAAT following α2,6 PTB treatment, the inventors were interested to see if the DSG activity of α2,6 PTB could be exploited to further increase the level of sialylation on the rAAT. First the amount of α2,3 sialylation already present on the rAAT needed to be increased. To achieve this, the rAAT was incubated with the α2,3 sialyltransferase from Pasteurella Multocida (α2,3PM) and GalT for 16 hr. α2,6PTB was added into the reaction mixture and incubated for a further 4 hr with rAAT. The resulting LC-MS/MS analysis of the tryptic glycopeptides showed large increases in the multiply sialylated glycans and average SA count rose to 6.6 for the rAAT (FIG. 1 & Table 1). This highlighted how a multi enzyme one pot reaction could be used to significantly increase the sialylation of rAAT by exploiting a2,6 PTB hypersialylation activity. It indicates how α2,6 PTB may be a useful tool for increasing highly α2,3 sialylated glycoproteins where further increasing sialylation has become difficult and/or impossible.

The inventors investigated whether the activity of the rAAT was negatively altered by the glycan remodelling process. Following purification and a ELISA elastase activity assay it was found that the activity before (89.3%) and after glycan remodelling of the rAAT (95.4%) was similar (Table 7). The enzyme treated rAAT having activity 95.4% is also comparable to native human plasma rAAT (100%). This suggest that the DSG sialylation and the incubation process has no significant effect on the activity of the remodeled rAAT.

To see if the DSG sialylation gave any sialidase resistance, the inventors incubated some of the tryptic glycopeptides of the α2,6PTB treated rAAT with ABS and subsequently analysed the glycopeptides by LC-MS/MS. The results of the sialidase testing showed that the DSG offered no apparent sialidase resistance with overall sialylation dropping from 93% to 2% (Table 8).

TABLE 1
Sialic Acid Count (SA Count) of rAAT following each incubation

| Enzymes AAT | |
|---|---|
| Incubated With | *SA Count |
| None | 2.6 |
| α2,6PTB | 3.3 |
| α2,6PTB + GalT | 3.8 |
| α2,3PM + GalT + α2,6PTB | 6.6 |

$* SA Count = \frac{( 1 \times \%1 SA ) + ( 2 \times \%2 SA ) + ( 3 \times \%3 SA ) + ( 4 \times \%4 SA ) + ( 5 \times \%5 SA )}{100} \times N$ Where: %1SA = % Glycans with 1 sialic acid, %2SA = % Glycans with 2 sialic acid . . . etc. N = number of glycosylation sites on a protein (for rAAT N = 3).

TABLE 2
Breakdown of the N-glycan composition observed during LC-MS/MS glycopeptide analysis
of the untreated rAAT. N.B. All values given are from triplicate analysis. All values
are calculated as the % contribution to the total glycan intensity observed in the
LC-MS/MS where the Total Glycan signal = 100%.
Untreated rAAT

| | Branching | | | | | | | | | | |
|---|---|---|---|---|---|---|---|---|---|---|---|
| | Relative | | Terminal | Relative | | Terminal | Relative | | | Relative | |
| | Glycans | | Galactose | Glycans | | GlcNAc | Glycans | | Neu5Ac | Glycans | |
| No. | (%) | SEM | No. | (%) | SEM | No. | (%) | SEM | No. | (%) | SEM |
| 0 | 3.14 | 0.54 | 1 | 31.15 | 1.65 | 1 | 15.13 | 0.24 | 1 | 21.44 | 1.12 |
| 1 | 2.15 | 0.38 | 2 | 21.21 | 0.77 | 2 | 23.83 | 1.66 | 2 | 16.84 | 1.66 |
| 2 | 60.22 | 1.56 | 3 | 4.41 | 0.75 | 3 | 3.40 | 1.32 | 3 | 7.52 | 1.31 |
| 3 | 14.79 | 0.72 | 4 | 0.81 | 0.17 | 4 | 0.86 | 0.22 | 4 | 1.84 | 0.69 |
| 4 | 13.83 | 0.34 | *5 | 0.25 | 0.23 | All | 43.23 | 3.35 | All | 47.64 | 3.26 |
| *5 | 5.13 | 0.55 | All | 57.84 | 0.54 | | | | | | |
| *6 | 0.75 | 0.25 | | | | | | | | | |
| All | 100.00 | | | | | | | | | | |

TABLE 3
Breakdown of the N-glycan composition observed during LC-MS/MS glycopeptide analysis
of the α2,6 sialyltransferase Photobacterium Damselae (α2,6PTB), treated rAAT.
N.B. All values given are from triplicate analysis. All values are calculated as the %
contribution to the total glycan intensity observed in the LC-MS/MS where the
Total Glycan signal = 100%.
rAAT + α2,6PTB

| | Branching | | | | | | | | | | |
|---|---|---|---|---|---|---|---|---|---|---|---|
| | Relative | | Terminal | Relative | | Terminal | Relative | | | Relative | |
| | Glycans | | Galactose | Glycans | | GlcNAc | Glycans | | Neu5Ac | Glycans | |
| No. | (%) | SEM | No. | (%) | SEM | No. | (%) | SEM | No. | (%) | SEM |
| 0 | 3.78 | 0.31 | 1 | 26.99 | 1.06 | 1 | 16.89 | 0.64 | 1 | 21.09 | 0.77 |
| 1 | 2.64 | 0.06 | 2 | 14.18 | 0.79 | 2 | 22.69 | 0.55 | 2 | 18.53 | 0.98 |
| 2 | 59.02 | 0.55 | 3 | 2.98 | 0.12 | 3 | 4.65 | 0.71 | 3 | 11.97 | 1.19 |
| 3 | 14.90 | 0.76 | 4 | 0.38 | 0.08 | 4 | 0.74 | 0.14 | 4 | 4.01 | 1.33 |
| 4 | 13.74 | 0.69 | All | 44.53 | 0.59 | All | 44.97 | 1.57 | 5 | 0.19 | 0.10 |
| *5 | 5.55 | 0.84 | | | | | | | All | 55.78 | 0.97 |
| *6 | 0.37 | 0.16 | | | | | | | | | |
| All | 100.00 | | | | | | | | | | |

TABLE 4
Breakdown of the N-glycan composition observed during LC-MS/MS glycopeptide analysis
of the α2,6 sialyltransferase Photobacterium Damselae (α2,6PTB), β1,4-galactosyltransferase
from Bovine Taurus milk (GalT) treated rAAT. N.B. All values given are from triplicate analysis.
All values are calculated as the % contribution to the total glycan intensity observed in the
LC-MS/MS where the Total Glycan signal = 100%.
rAAT + GalT + α2,6PTB

| | Branching | | | | | | | | | | |
|---|---|---|---|---|---|---|---|---|---|---|---|
| | Relative | | Terminal | Relative | | Terminal | Relative | | | Relative | |
| | Glycans | | Galactose | Glycans | | GlcNAc | Glycans | | Neu5Ac | Glycans | |
| No. | (%) | SEM | No. | (%) | SEM | No. | (%) | SEM | No. | (%) | SEM |
| 0 | 3.73 | 0.43 | 1 | 36.86 | 3.24 | 1 | 0.46 | 0.13 | 1 | 38.03 | 1.79 |
| 1 | 2.29 | 0.04 | 2 | 25.32 | 2.92 | 2 | 0.03 | 0.01 | 2 | 20.71 | 0.24 |
| 2 | 58.62 | 0.40 | 3 | 9.47 | 0.36 | 3 | 0.21 | 0.06 | 3 | 9.74 | 0.95 |
| 3 | 17.10 | 0.65 | 4 | 2.68 | 0.47 | 4 | 0.14 | 0.01 | 4 | 3.38 | 0.45 |
| 4 | 13.57 | 1.05 | *5 | 0.20 | 0.17 | All | 0.83 | 0.07 | 5 | 0.65 | 0.05 |
| *5 | 4.40 | 0.44 | All | 74.53 | 1.00 | | | | All | 72.51 | 2.88 |
| *6 | 0.29 | 0.07 | | | | | | | | | |
| All | 100.00 | | | | | | | | | | |

TABLE 5
Breakdown of the N-glycan composition observed during LC-MS/MS glycopeptide
analysis of the α2,6 sialyltransferase Photobacterium Damselae (α2,6PTB), β1,4-
galactosyltransferase from Bovine Taurus milk (GalT) and α2,3 sialyltransferase
from Pasteurella multocida (α2,3PM) treated rAAT. N.B. All values given are
from triplicate analysis. All values are calculated as the % contribution to the total glycan
signal intensity observed in the LC-MS/MS where the Total Glycan signal = 100%.
rAAT + GalT + α2,3PM + α2,6PTB

| | Branching | | | | | | | | | | |
|---|---|---|---|---|---|---|---|---|---|---|---|
| | Relative | | Terminal | Relative | | Terminal | Relative | | | Relative | |
| | Glycans | | Galactose | Glycans | | GlcNAc | Glycans | | Neu5Ac | Glycans | |
| No. | (%) | SEM | No. | (%) | SEM | No. | (%) | SEM | No. | (%) | SEM |
| 0 | 5.57 | 0.31 | 1 | 29.46 | 1.75 | 1 | 0.11 | 0.01 | 1 | 19.58 | 2.48 |
| 1 | 2.11 | 0.03 | 2 | 9.51 | 0.68 | 2 | 0.00 | 0.00 | 2 | 33.42 | 2.89 |
| 2 | 49.07 | 1.43 | 3 | 3.34 | 0.29 | 3 | 0.00 | 0.00 | 3 | 27.54 | 0.91 |
| 3 | 18.65 | 0.30 | 4 | 0.18 | 0.06 | 4 | 0.04 | 0.04 | 4 | 11.18 | 0.64 |
| 4 | 21.00 | 1.06 | All | 42.49 | 2.32 | All | 0.15 | 0.04 | 5 | 1.30 | 0.25 |
| *5 | 3.39 | 0.22 | | | | | | | All | 93.02 | 0.50 |
| *6 | 0.21 | 0.02 | | | | | | | | | |
| 100 | 100.00 | | | | | | | | | | |

*Indicates presence of Di-LacNAc structures.

TABLE 6
Table showing all the permethylated N-Glycans of α2,6 sialyltransferase
Photobacterium Damselae treated rAAT observed in the MALDI-TOF MS.
Permethylated N-Glycan observed in MALDI-TOF-MS of α2,6PTB treated rAAT

| [M + Na]⁺ | Permethylated N-Glycan | [M + Na]⁺ | Permethylated N-Glycan |
|---|---|---|---|
| 1171.52 | 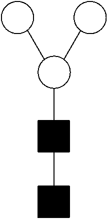 | 1345.67 | 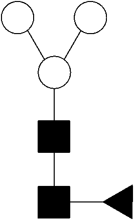 |
| 1579.78 | 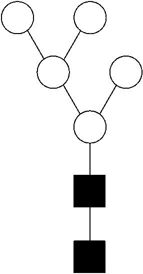 | 1783.89 | 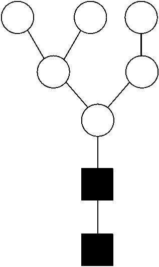 |
| 1794.90 | 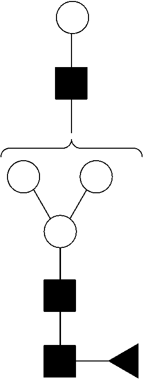 | 2070.02 | 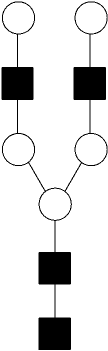 |
| 2156.07 | 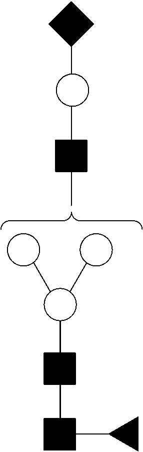 | 2244.11 | 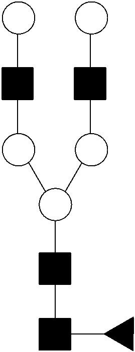 |
| 2431.21 | 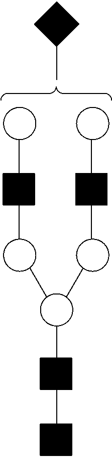 | 2517.24 | 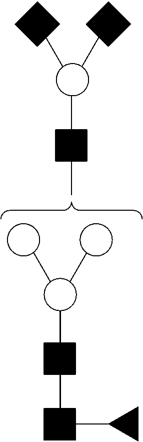 |
| 2605.29 | 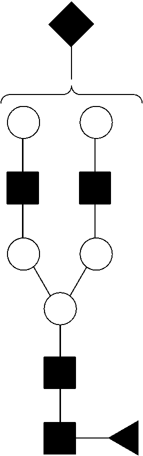 | 2693.3 | 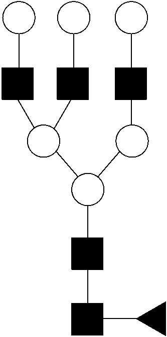 |
| 2792.36 | 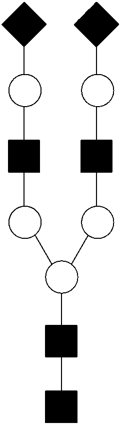 | 2880.42 | 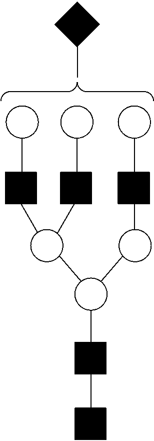 |
| 2966.49 | 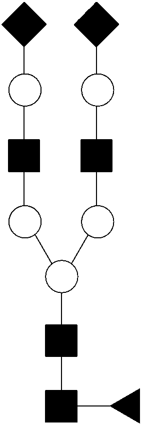 | 3054.49 | 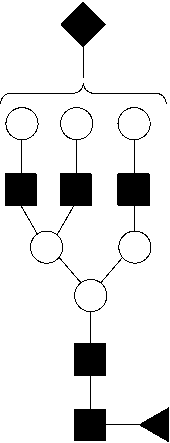 |
| 3327.61 | 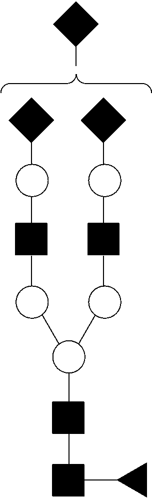 | 3415.66 | 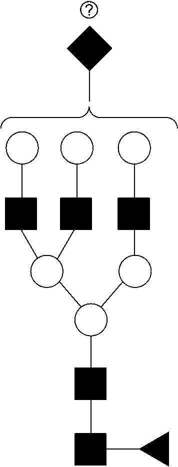 |
| 3503.70 | 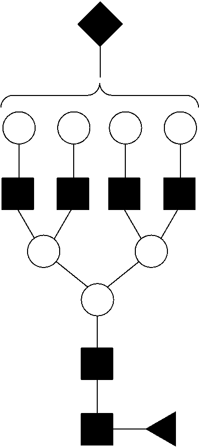 | 3688.75 | 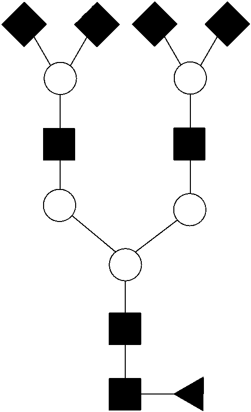 |
| 3776.81 | 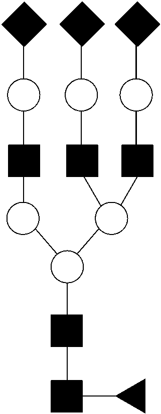 | 3864.86 | 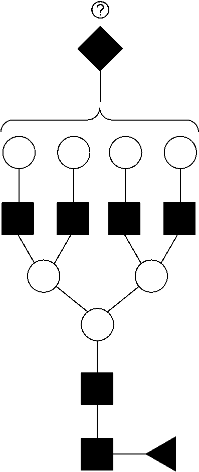 |
| 4138.10 | 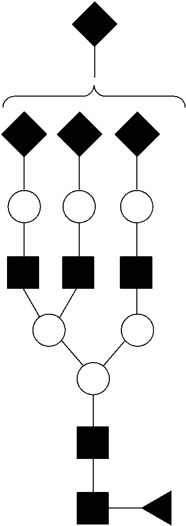 | 4226.02 | 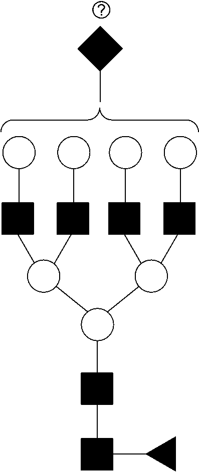 |
| 4314.01 | 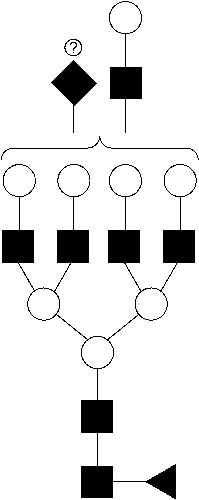 | 4587.27 | 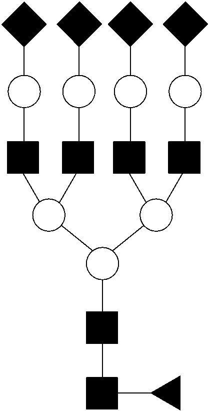 |

| | |
|---|---|
| 4675.21 | 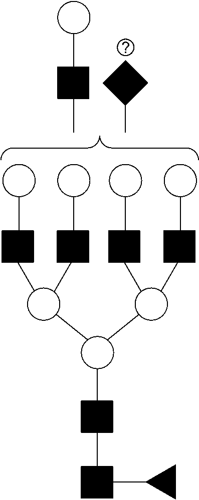 |

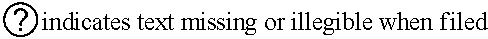

TABLE 7
Breakdown of the N-glycan Neu5Ac levels observed during
LC-MS/MS glycopeptide analysis following sialidase incubation
using Arthrobacter ureafaciens ABS on the tryptic
digested α2,6PTB treated rAAT glycopeptides. N.B. All
values are calculated as the % contribution to the total glycan
signal intensity observed in the LC-MS/MS where the
Total Glycan signal = 100%.

Neu5Ac

| | Relative Glycans | Relative Glycans |
|---|---|---|
| | Before ABS | After ABS |
| | Treatment | Treatment |
| No. | (%) | (%) |
| 1 | 19.58 | 1.54 |
| 2 | 33.42 | 0.16 |
| 3 | 27.54 | 0.00 |
| 4 | 11.18 | 0.00 |
| 5 | 1.30 | 0.00 |
| All | 93.02 | 1.71 |

TABLE 8
Activity assay for remodelled rAAT before and after glycan
remodelling with α2,6PTB as well as a buffer control.

| Sample | | | Average % |
|---|---|---|---|
| Name | Assay 1 | Assay 2 | Activity |
| Remodelled | 97.1 | 93.0 | 95.4 |
| rAAT | | | |
| rAAT | 90.1 | 88.6 | 89.3 |
| Buffer | <LD | <LD | <LD |

1. An in vitro method, the method comprising a step of incubating an alpha 2,3 sialylated glycoprotein with an alpha 2,6 sialyltransferase and a sialic acid source for a sufficient time and under conditions to increase the number of α2,3,-α2,6-disialylgalactose (Neu5Ac-α2,3(Neu5Ac-α2,6)Gal) N-glycans on the glycoprotein as compared to a glycoprotein that has not been incubated with the alpha 2,6 sialyltransferase and the sialic acid source.

2. The method of claim 1, wherein the method comprises improving the pharmacokinetics of the glycoprotein.

3. The method of claim 2, wherein the method comprises improving the in vivo half-life of the therapeutic glycoprotein.

4. The method of claim 1, wherein the sialic acid source is cytidine-monophosphate-N-Acetyl-Neuraminic-Acid.

5. The method of claim 1, wherein the α2,6 sialyltransferase is an α2,6 sialyltransferase from a photobacterium.

6. The method of claim 5, wherein the α2,6 sialyltransferase is a purified α2,6 sialyltransferase from photobacterium or is an α2,6 sialyltransferase enzyme extract from photobacterium.

7. The method of claim 5, wherein the photobacterium is Photobacterium damselae.

8. The method of claim 1, wherein the glycoprotein is a recombinant glycoprotein or an isolated naturally-occurring glycoprotein.

9. The method of claim 8, wherein the glycoprotein is a Chinese Hamster Ovary (CHO) cell expressed glycoprotein.

10. The method of claim 1, wherein the glycoprotein is alpha-1 antitrypsin (AAT).

11. The method of claim 1, wherein the method comprises a prior or concurrent step of incubating the glycoprotein with an alpha 2,3 sialyltransferase and a sialic acid source for a sufficient time and under conditions to increase alpha 2,3 sialylation of the glycoprotein to a saturation point as compared to a glycoprotein that has not been incubated with an alpha 2,3 sialyltransferase and a sialic acid source.

12. The method of claim 1, wherein the method comprises a prior or concurrent step of incubating the glycoprotein with a β-1,4-galactosyltransferase and a galactose source for a sufficient time and under conditions to increase branching, the elongation and/or galactosylation of the glycoprotein as compared to a glycoprotein that has not been incubated with the β-1,4-galactosyltransferase and a galactose source.

13. A method of increasing sialylation of a glycoprotein, the method comprising a step of incubating an alpha 2,3 sialylated glycoprotein with an alpha 2,6 sialyltransferase and a sialic acid source for a sufficient time and under conditions to increase the number of α2,3,-α2,6-disialylgalactose N-glycans on the glycoprotein as compared to a glycoprotein that has not been incubated with the alpha 2,6 sialyltransferase and the sialic acid source.

14. A glycoprotein obtained according to a method of claim 1.

15. The glycoprotein of claim 14, wherein the glycoprotein comprises at least one α2,3,-α2,6-disialylgalactose N-glycan.

16. The glycoprotein of claim 15, wherein the recombinant glycoprotein comprises an amino acid sequence of SEQ ID NO: 4, wherein the amino acid sequence of SEQ ID NO:4 comprises an α2,3,-α2,6-disialylgalactose N-glycan at an amino acid position selected from the group consisting of Asn-46, Asn-83 and Asn-247.

17-23. (canceled)
